(19) United States
(12) Patent Application Publication
(10) Pub. No.: US 2016/0361456 A1
(43) Pub. Date: Dec. 15, 2016

(54) BIOMEDICAL AND PHARMACEUTICAL WASTE STERILIZING SYSTEMS AND METHODS WITH OZONE INJECTION

(71) Applicant: Richard F. ADMANI, Charlotte, NC (US)

(72) Inventor: Richard F. ADMANI, Charlotte, NC (US)

(73) Assignee: ENVIRONTEK WORLDWIDE, LLC, Charlotte, NC (US)

(21) Appl. No.: 14/823,570

(22) Filed: Aug. 11, 2015

Related U.S. Application Data
(63) Continuation-in-part of application No. 14/734,459, filed on Jun. 9, 2015.

(51) Int. Cl. A61L 11/00; B09B 3/00; B02C 19/00; A62D 3/38

(52) U.S. Cl. CPC A61L 11/00; A62D 3/38; B09B 3/0075; B02C 19/0075

(57) ABSTRACT

A biomedical and pharmaceutical waste sterilization system with an ozone injection system includes a vessel including a treatment chamber, wherein the vessel is disposed in an open frame for support thereof and the vessel includes an opening on a top side configured to receive biomedical waste and a shredding system to treat the biomedical waste; and a circulation loop configured to provide water and ozone injection into the water into the treatment chamber and to receive water from the treatment chamber during a treatment cycle for the biomedical waste.

### CROSS-REFERENCE TO RELATED APPLICATION(S)

The present patent/application is a continuation-in-part of U.S. patent application Ser. No. 14/734,459, filed Jun. 9, 2015, and entitled “BIOMEDICAL AND PHARMACEUTICAL WASTE STERILIZING SYSTEMS AND METHODS,” the contents of which are incorporated by reference.

### FIELD OF THE DISCLOSURE

The present disclosure generally relates to waste remediation systems and methods. More particularly, the present disclosure relates to biomedical and pharmaceutical waste sterilizing systems and methods with ozone injection.

### BACKGROUND OF THE DISCLOSURE

Biomedical waste includes, for example, discarded blood, unwanted microbiological cultures and stocks, identifiable body parts, other human or animal tissue, used bandages and dressings, discarded gloves, other medical supplies that may have been in contact with blood and body fluids, laboratory waste that exhibits the characteristics described above, pharmaceutical compounds such as expired or discarded drugs, etc. Biomedical waste is generated from biological and medical sources and activities, such as the diagnosis, prevention, or treatment of diseases. Common generators (or producers) of biomedical waste include hospitals, health clinics, nursing homes, medical research laboratories, offices of physicians, dentists, and veterinarians, home health care, and the like. It is important to remediate or treat biomedical waste. Conventional biomedical waste sterilizing systems and methods are physically large, capital intensive, and, for ozone treatment, require on-site ozone generation. There is a need to address the aforementioned limitations in conventional biomedical waste sterilizing systems and methods.

### BRIEF SUMMARY OF THE DISCLOSURE

In an exemplary embodiment, a biomedical and pharmaceutical waste sterilization system with an ozone injection system includes a vessel including a treatment chamber, wherein the vessel is disposed in an open frame for support thereof and the vessel includes an opening on a top side configured to receive biomedical waste and a shredding system to treat the biomedical waste; and a circulation loop configured to provide water and ozone injection into the water into the treatment chamber and to receive water from the treatment chamber during a treatment cycle for the biomedical waste. The circulation loop can include an ozone generator coupled to an ozone injector coupled to the treatment chamber; a water pump coupled to the ozone injector; a return pipe from the treatment chamber into the water pump, wherein the circulation loop runs from the treatment chamber to the water pump to the ozone injector and back to the treatment chamber. The circulation loop can further include a water filter between the water pump and the treatment chamber. Ozone can be provided to treat waste in the treatment chamber by one or more of injecting ozone into water through the circulation loop, recirculating already ozone infused water through the circulation loop, and injecting ozone gas into water before introducing the water into the treatment chamber. An amount of ozone provided for the treatment can be determined by a control system based on feedback from one of an Oxidation-Reduction Potential (ORP) meter or sensor and a pH meter monitoring water from the treatment chamber.

The vessel can be configured to receive the biomedical waste and to treat the biomedical waste in a top loading position, and wherein the vessel is further configured to tilt after the treatment cycle to remove treated biomedical waste. The vessel can be configured to tilt using a gear system coupled to the vessel. The biomedical and pharmaceutical waste sterilization system can further include a cover rotatably connected to a member of the frame and configured to cover the opening in the vessel during the treatment cycle and non-use and to allow access to the opening during loading of the biomedical waste. The cover may not be physically attached or connected to the vessel. The shredding system can include a cylinder with a plurality of cutting teeth disposed thereon and a gear system configured to rotate the cylinder, and wherein the cylinder is located in a bottom portion of the vessel. The gear system can be configured to rotate the cylinder both clockwise and counter-clockwise at variable speeds, based on feedback from a control system associated with the biomedical waste sterilization system. The biomedical and pharmaceutical waste sterilization system can further include a control system configured to monitor and control operation of the biomedical waste sterilization system, the control system including a verification/billing component, a shredder control component, and a User Interface. The control system can be configured to operate the shredder control component based on feedback from one of an Oxidation-Reduction Potential (ORP) meter or sensor and a pH meter.

In another exemplary embodiment, a biomedical and pharmaceutical waste sterilization method with an ozone injection system includes providing a vessel including a treatment chamber, wherein the vessel is disposed in an open frame for support thereof and the vessel includes an opening on a top side configured to receive biomedical waste and a shredding system to treat the biomedical waste; and providing a circulation loop configured to provide water and ozone injection into the water into the treatment chamber and to receive water from the treatment chamber during a treatment cycle for the biomedical waste. The circulation loop can include an ozone generator coupled to an ozone injector coupled to the treatment chamber; a water pump coupled to the ozone injector; a return pipe from the treatment chamber into the water pump, wherein the circulation loop runs from the treatment chamber to the water pump to the ozone injector and back to the treatment chamber. The circulation loop can further include a water filter between the water pump and the treatment chamber. Ozone is provided to treat waste in the treatment chamber by one or more of injecting ozone into water through the circulation loop, recirculating already ozone infused water through the circulation loop, and injecting ozone gas into water before introducing the water into the treatment chamber. An amount of ozone provided for the treatment can be determined by a control system based on feedback from one of an Oxidation-Reduction Potential (ORP) meter or sensor and a pH meter monitoring water from the treatment chamber.

In a further exemplary embodiment, an apparatus includes a frame including a plurality of members for support and each side of the frame is substantially open; a vessel including a treatment chamber disposed in the frame for support thereof and the vessel includes an opening on a top side configured to receive biomedical waste and a shredding system to treat the biomedical waste; a circulation loop configured to provide water and ozone injection into the water into the treatment chamber and to receive water from the treatment chamber during a treatment cycle for the biomedical waste; a cover rotatably connected to a member of the frame and configured to cover the opening in the vessel during the treatment cycle and non-use and to allow access to the opening during loading of the biomedical waste; and a control system configured to monitor and control operation of the biomedical waste sterilization system, the control system including a verification/billing component, a shredder control component, and a User Interface. The circulation loop can include an ozone generator coupled to an ozone injector coupled to the treatment chamber; a water pump coupled to the ozone injector; a return pipe from the treatment chamber into the water pump, wherein the circulation loop runs from the treatment chamber to the water pump to the ozone injector and back to the treatment chamber.

In an exemplary embodiment, a biomedical waste sterilization system includes a vessel including a treatment chamber, wherein the vessel is disposed in an open frame for support thereof and the vessel includes an opening on a top side configured to receive biomedical waste and a shredding system to treat the biomedical waste; and a chemical loading system configured to receive liquid ozone for introduction into the treatment chamber prior to a treatment cycle for the biomedical waste.

In another exemplary embodiment, a biomedical waste sterilization method includes providing a vessel including a treatment chamber, wherein the vessel is disposed in an open frame for support thereof and the vessel includes an opening on a top side configured to receive biomedical waste and a shredding system to treat the biomedical waste; and providing a chemical loading system configured to receive liquid ozone for introduction into the treatment chamber prior to a treatment cycle for the biomedical waste.

In a further exemplary embodiment, an apparatus includes a frame including a plurality of members for support and each side of the frame is substantially open; a vessel including a treatment chamber disposed in the frame for support thereof and the vessel includes an opening on a top side configured to receive biomedical waste and a shredding system to treat the biomedical waste; a chemical loading system configured to receive liquid ozone for introduction into the treatment chamber prior to a treatment cycle for the biomedical waste; a cover rotatably connected to a member of the frame and configured to cover the opening in the vessel during the treatment cycle and non-use and to allow access to the opening during loading of the biomedical waste; and a control system configured to monitor and control operation of the biomedical waste sterilization system, the control system including a verification/billing component, a shredder control component, and a User Interface.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a front perspective view of a biomedical waste sterilizing system with all components included and with a cover closed;

FIG. 2 is a front perspective view of the biomedical waste sterilizing system with all components included and with the cover closed, illustrating an opposite side as FIG. 1;

FIG. 3 is a front perspective view of the biomedical waste sterilizing system with the cover removed;

FIG. 4 is a cross-sectional view of the biomedical waste sterilizing system in FIG. 2 showing an X-Z plane view;

FIG. 5 is a cross-sectional view of the biomedical waste sterilizing system in FIG. 2 showing an X-Z plane view, illustrating an opposite side as FIG. 4;

FIG. 6 is a front perspective view of the biomedical waste sterilizing system with the cover removed and excluding a frame;

FIG. 7 is a front perspective view of the frame of the biomedical waste sterilizing system;

FIG. 8 is a rear perspective view of the biomedical waste sterilizing system with all components included and with a cover closed;

FIG. 9 is a cross-sectional view of the biomedical waste sterilizing system showing an X-Z plane view, illustrating a vessel and chemical loading system;

FIG. 10 is a cross-sectional view of the biomedical waste sterilizing system showing an X-Y plane view;

FIG. 11 is a cross-sectional view of the biomedical waste sterilizing system showing a Y-Z plane view;

FIG. 12 is a perspective view of the vessel and a drawer of the biomedical waste sterilizing system;

FIG. 13 is a top view of the biomedical waste sterilizing system with the cover removed;

FIG. 14 is a side view of the biomedical waste sterilizing system with the cover in an open position;

FIGS. 15A-15B are perspective views of a liquid ozone capsule and a liquid ozone drum for use in the biomedical waste sterilizing system;

FIG. 16 is a block diagram of functional operation of biomedical waste sterilizing system;

FIG. 17 is a flowchart of a deployment and operational process of the biomedical waste sterilizing system;

FIG. 18 is a front perspective view of the biomedical waste sterilizing system with load cells configured to determine weight of the biomedical waste;

FIG. 19 is a block diagram of an integrated circuit (IC)-based control system for use with the biomedical waste sterilizing system;

FIG. 20 is a block diagram functionally illustrates a biomedical waste sterilizing system 300 with ozone injection.

### DETAILED DESCRIPTION OF THE DISCLOSURE

In various exemplary embodiments, biomedical and pharmaceutical waste sterilizing systems and methods with ozone injection are described. Particularly, the systems and methods utilize a compact apparatus which includes an on-site ozone generator, which is infused in a liquid. The compact apparatus is configured to properly sterilize biomedical and pharmaceutical waste based on monitoring during treatment and feedback providing additional ozone/treatment as required to properly treat the waste. In other various exemplary embodiments, biomedical waste sterilizing systems and methods are described. Particularly, the systems and methods utilize a compact apparatus that is (i) low cost to deploy and operate and (ii) that uses liquid ozone for sterilization/disinfection, while providing better sterilization/disinfection than conventional systems and methods. The cost reduction is based both on a compact design that minimizes capital deployment costs as well as the use of advanced liquid oxidation technology, avoiding on-site ozone generation equipment.

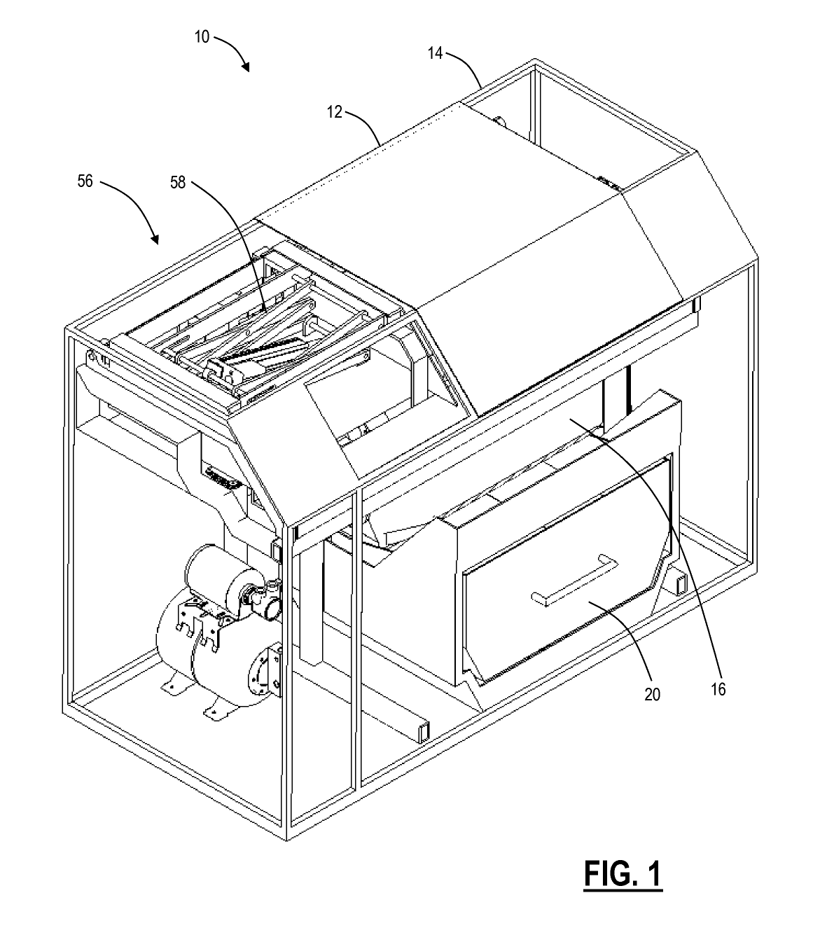

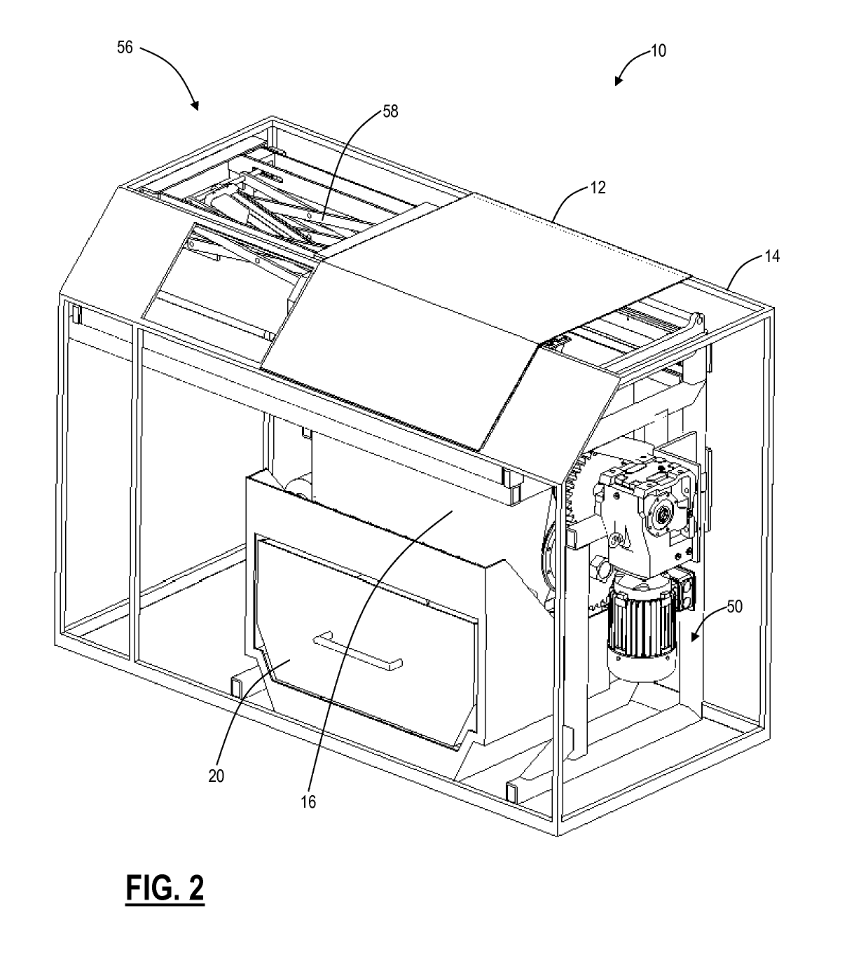

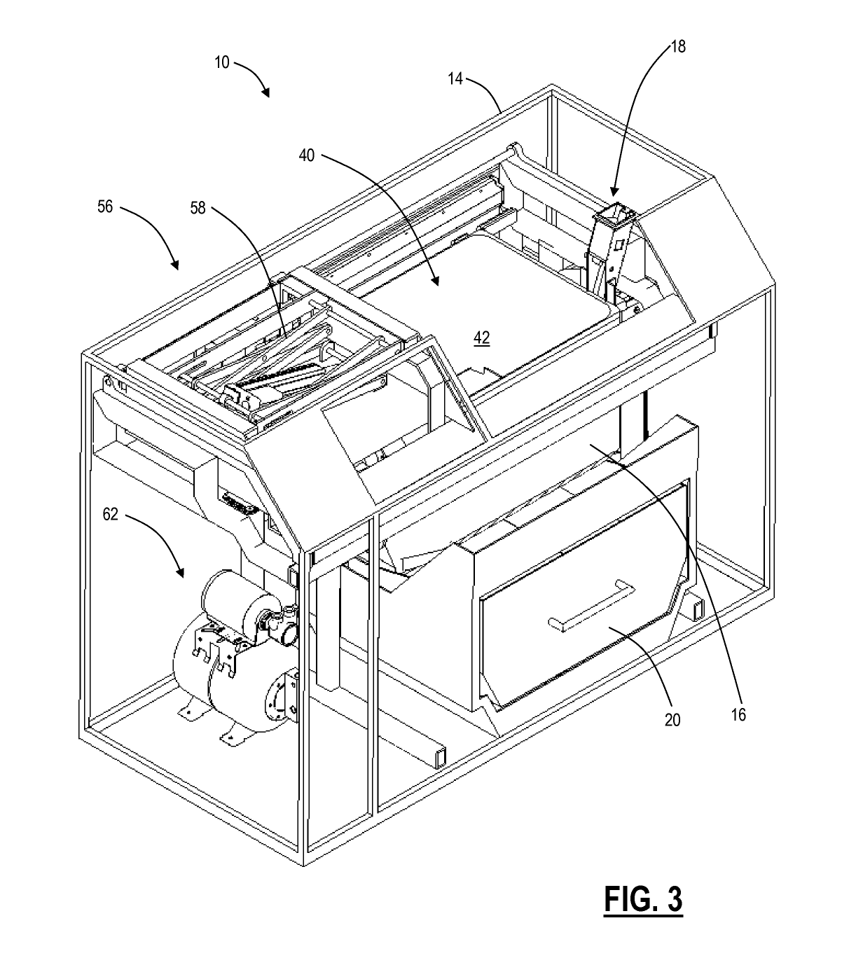

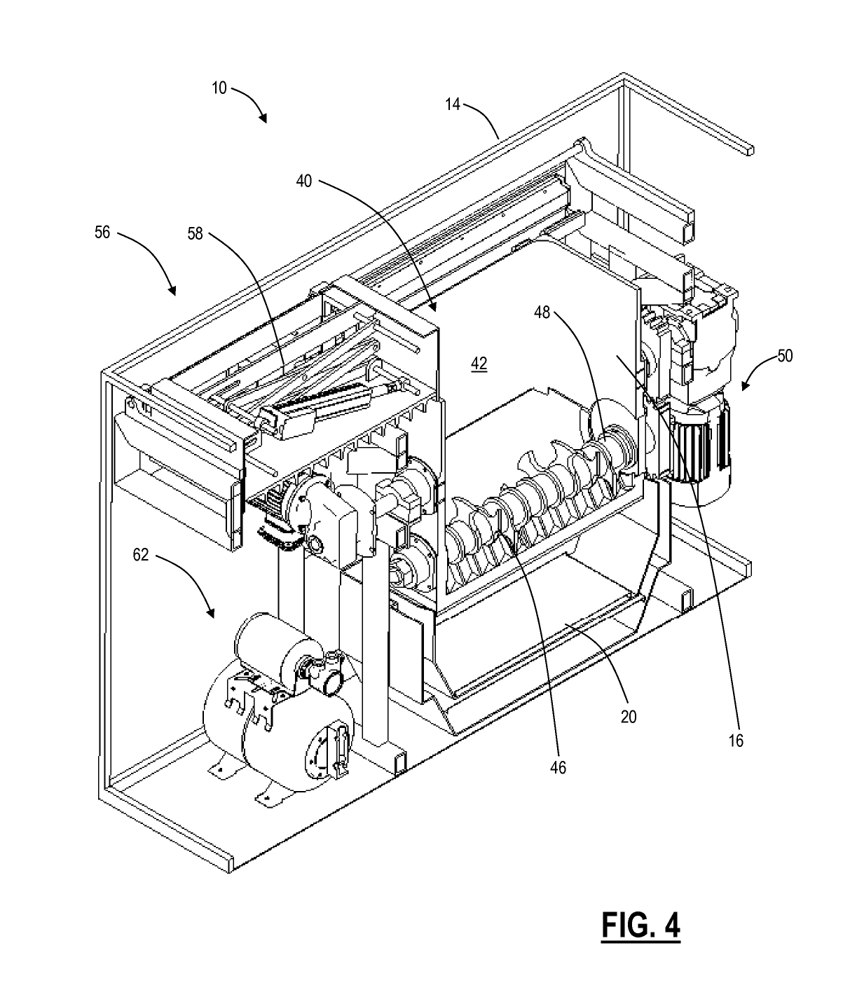

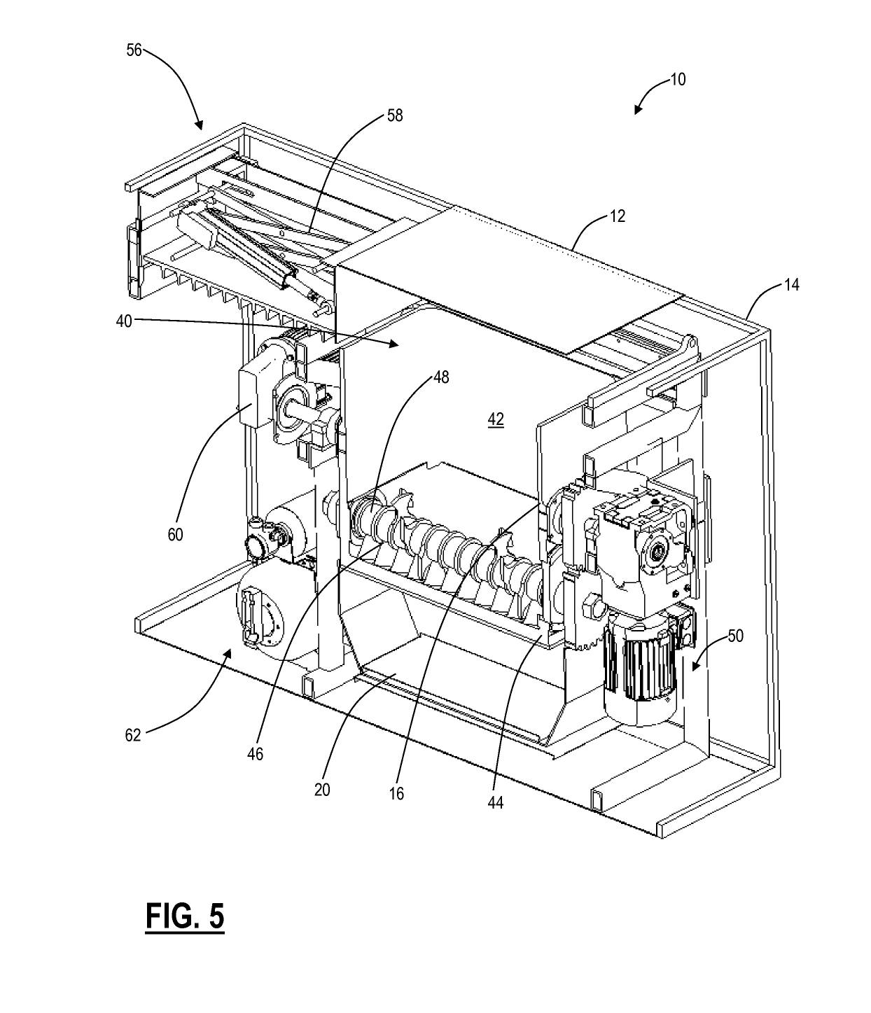

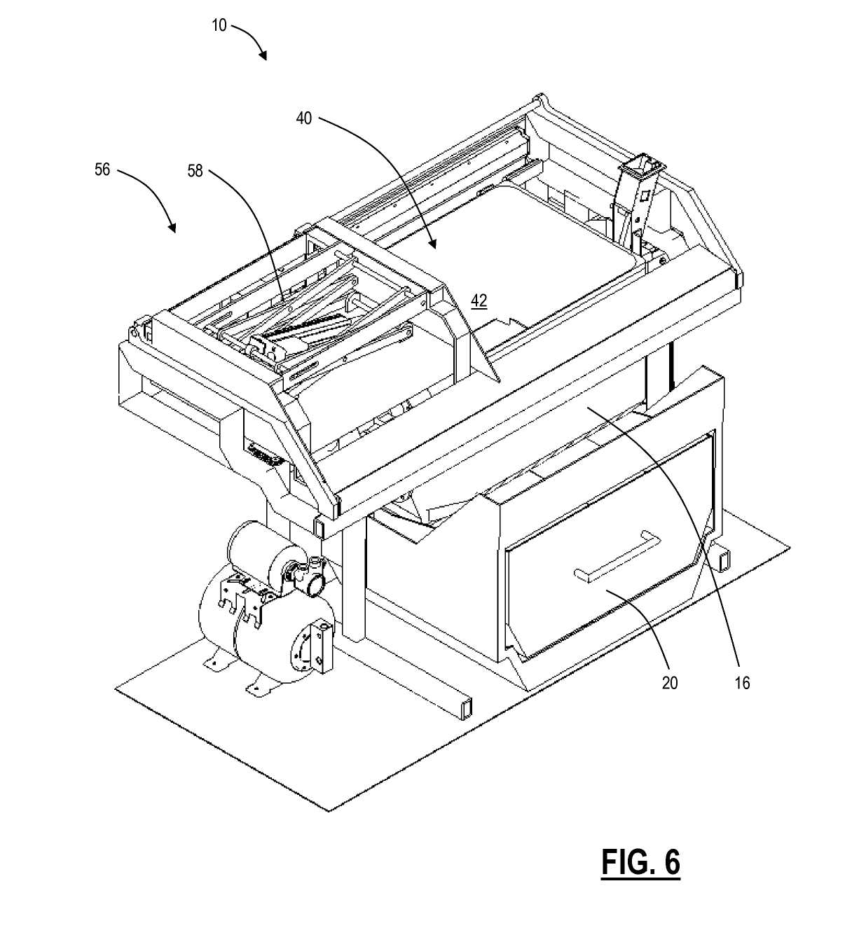

Referring to FIGS. 1-14, in various exemplary embodiments, various diagrams illustrate a biomedical waste sterilizing system 10. FIG. 1 is a front perspective view of the biomedical waste sterilizing system 10 with all components included and with a cover 12 closed. FIG. 2 is a front perspective view of the biomedical waste sterilizing system 10 with all components included and with the cover 12 closed, illustrating an opposite side as FIG. 1. FIG. 3 is a front perspective view of the biomedical waste sterilizing system with the cover 12 removed. FIG. 4 is a cross-sectional view of the biomedical waste sterilizing system 10 showing an X-Z plane view. FIG. 5 is a cross-sectional view of the biomedical waste sterilizing system 10 showing an X-Z plane view, illustrating an opposite side of FIG. 4. FIG. 6 is a front perspective view of the biomedical waste sterilizing system 10 with the cover 12 removed and excluding a frame 14.

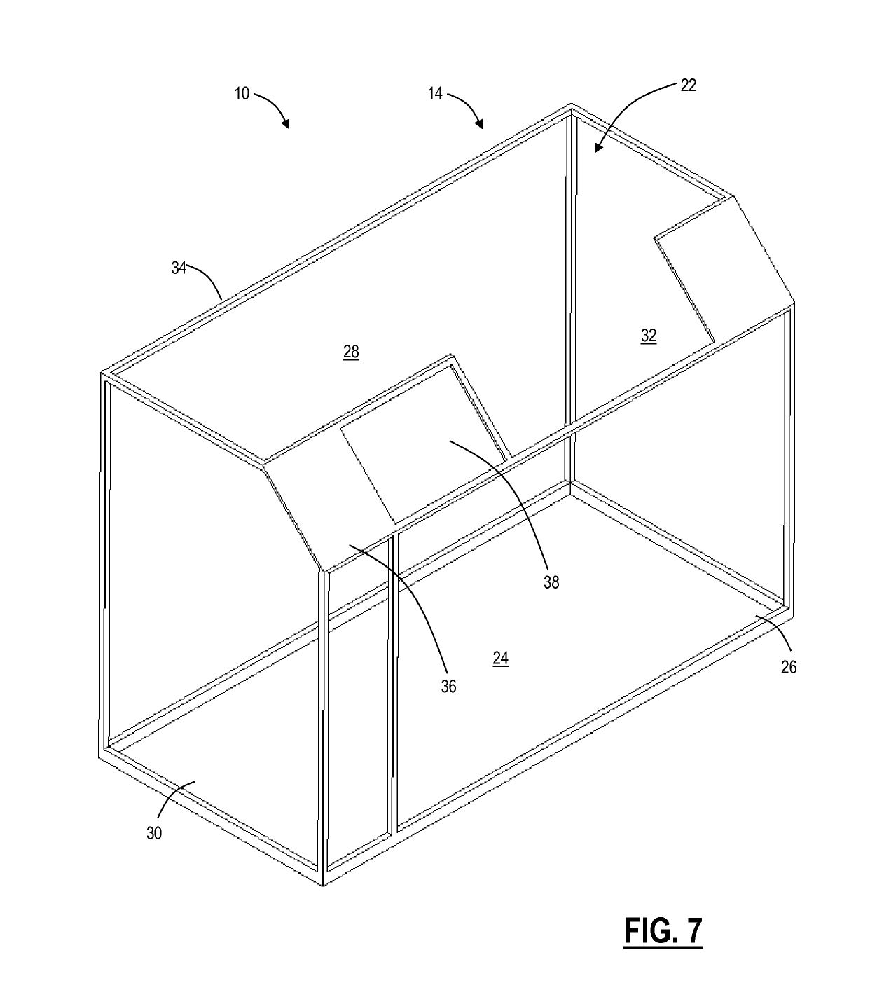

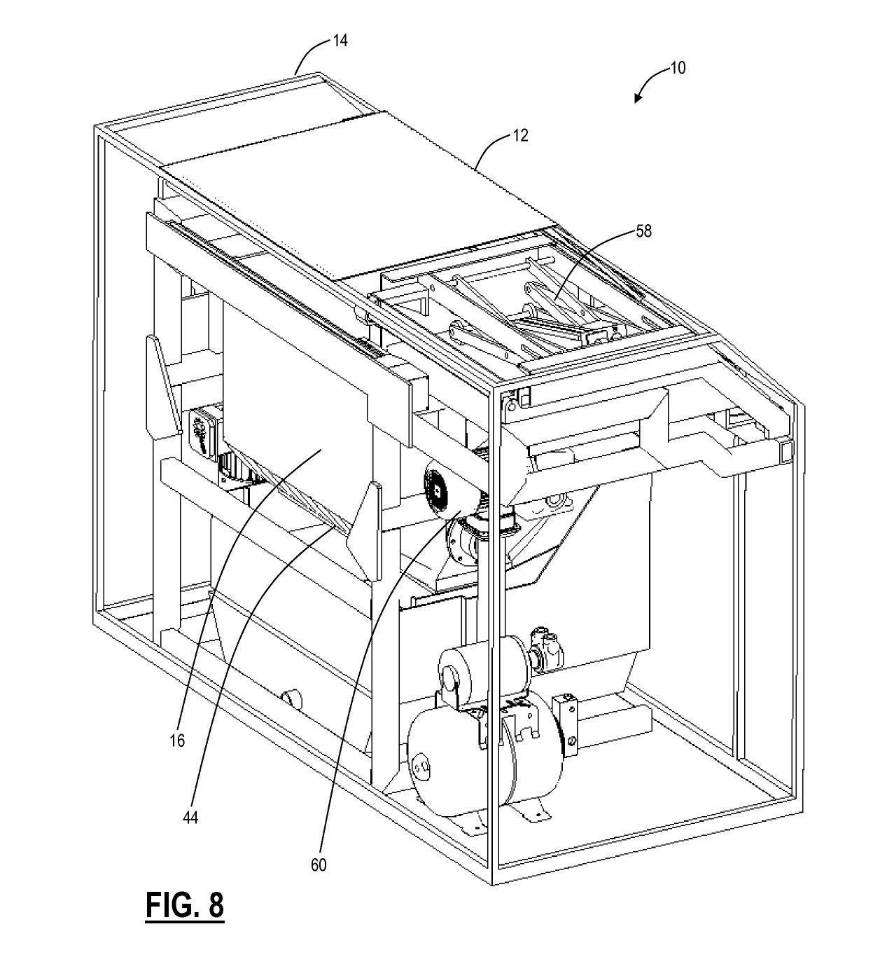

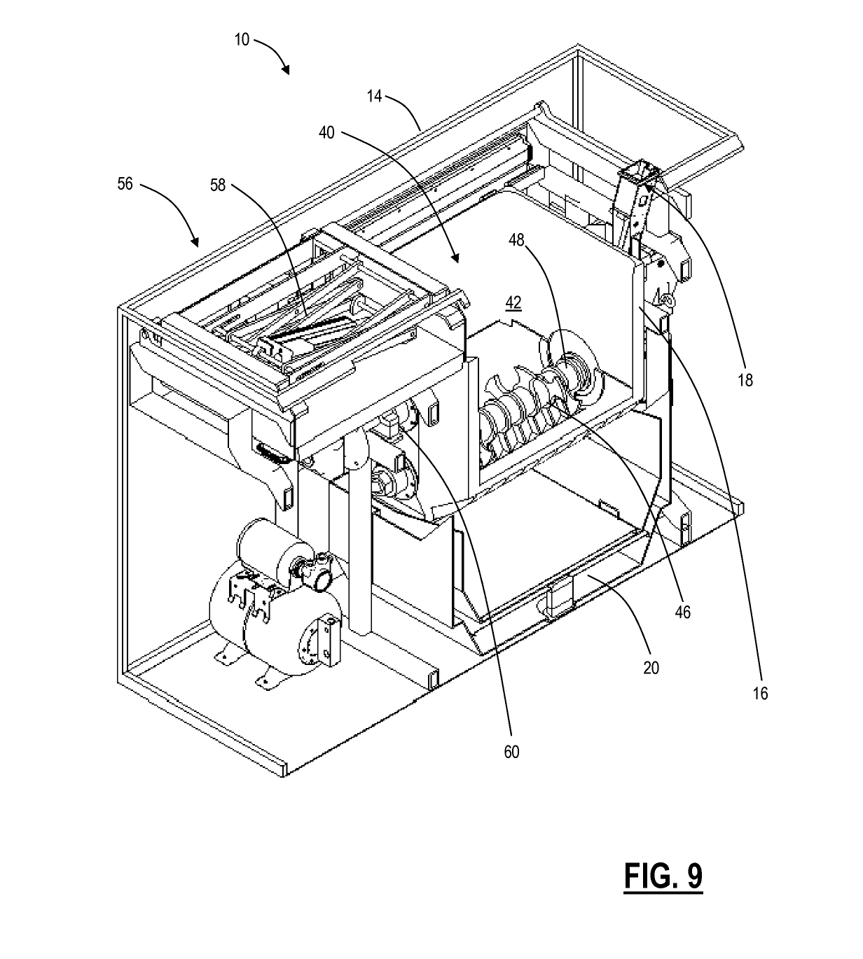

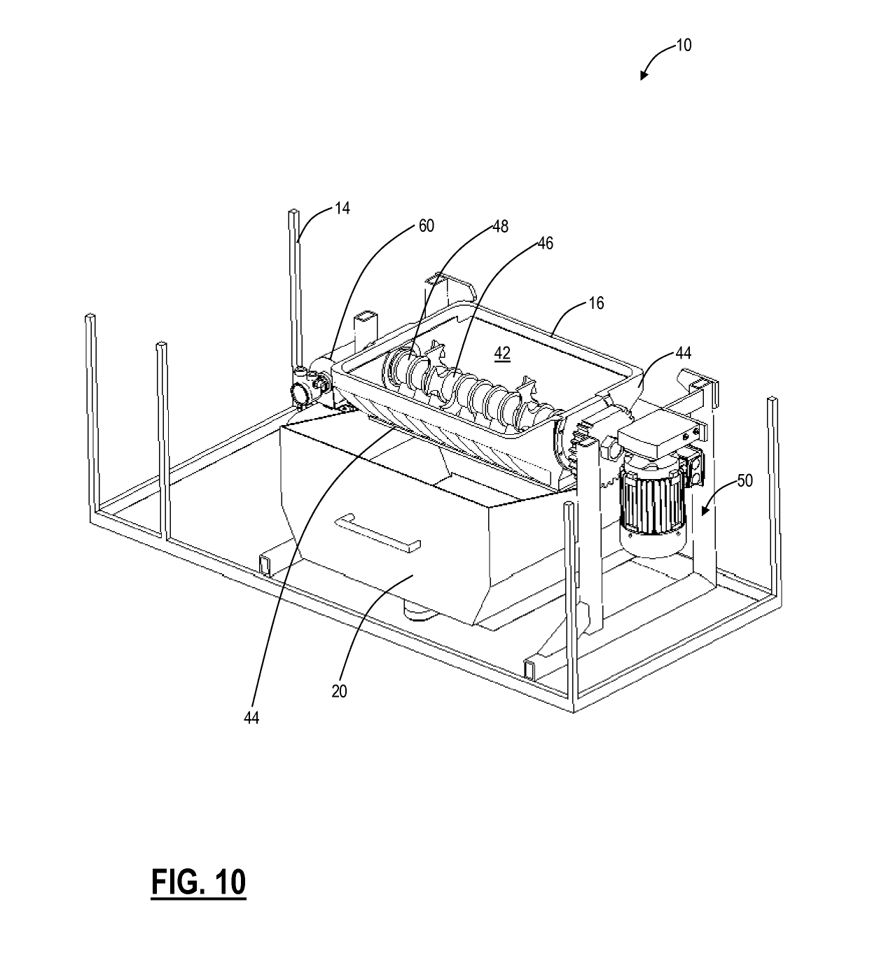

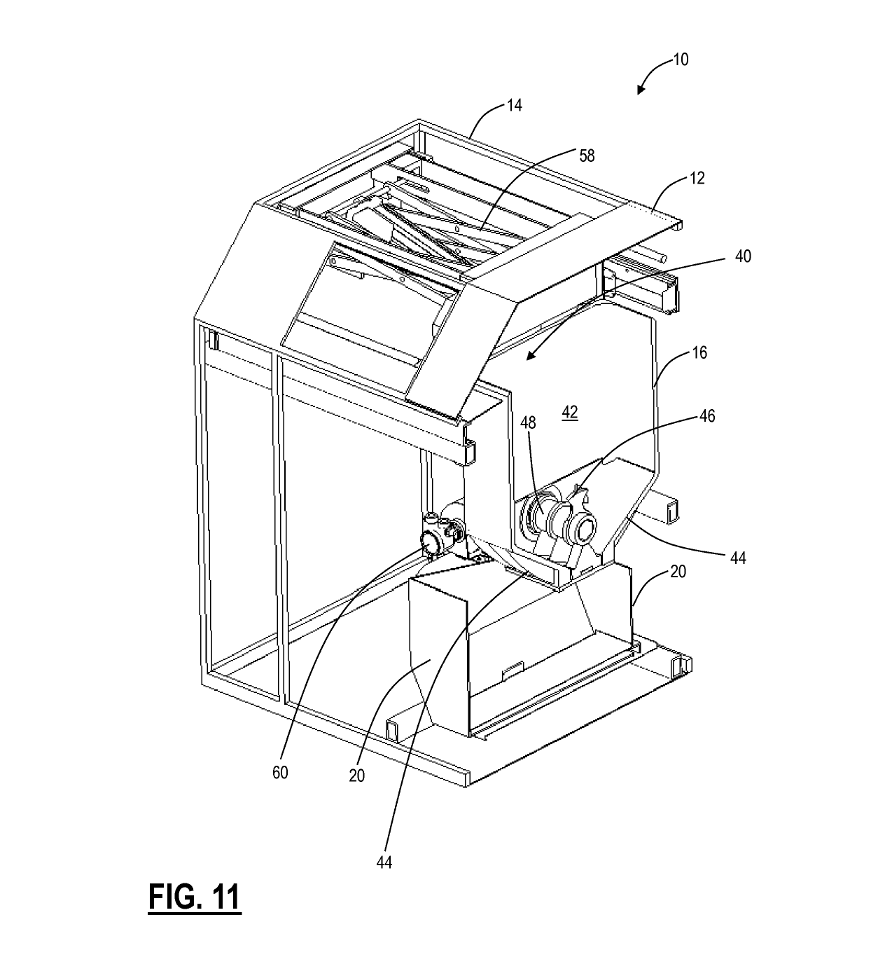

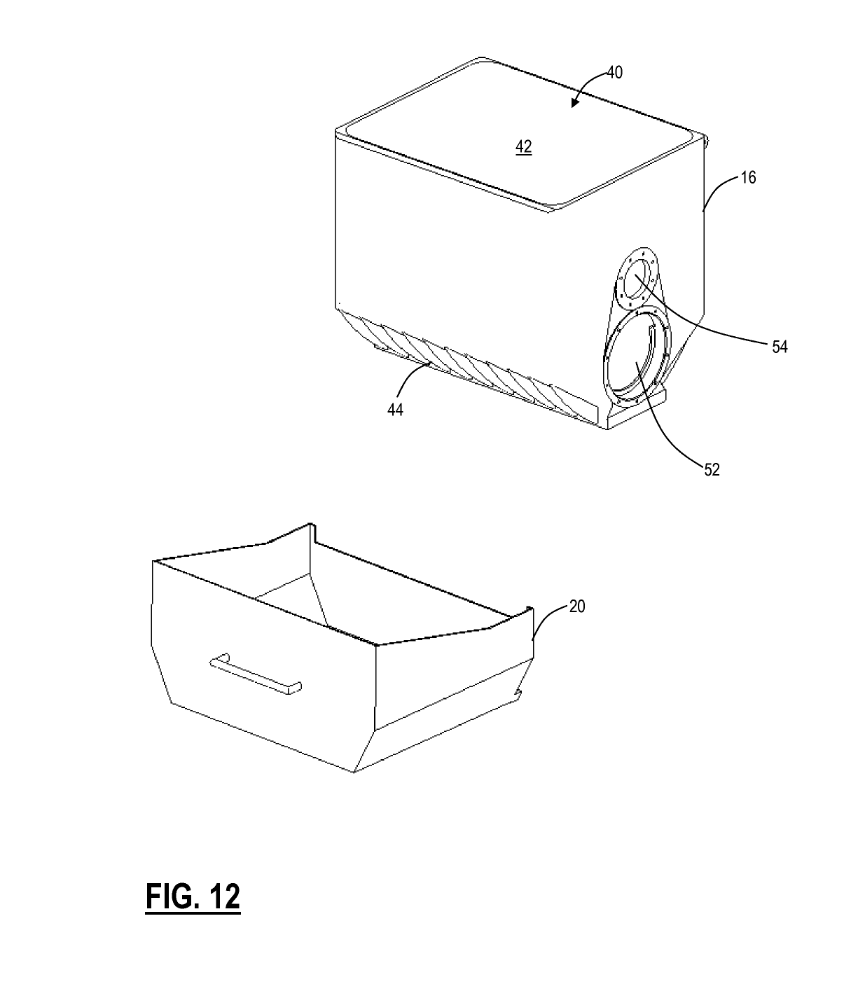

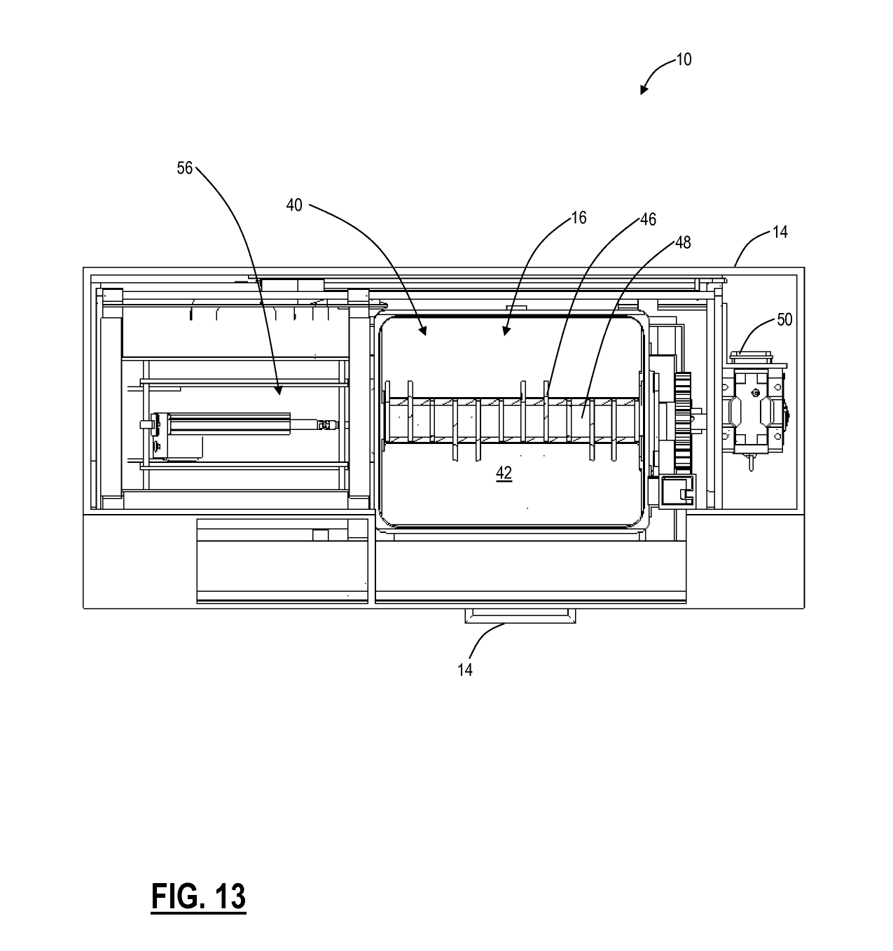

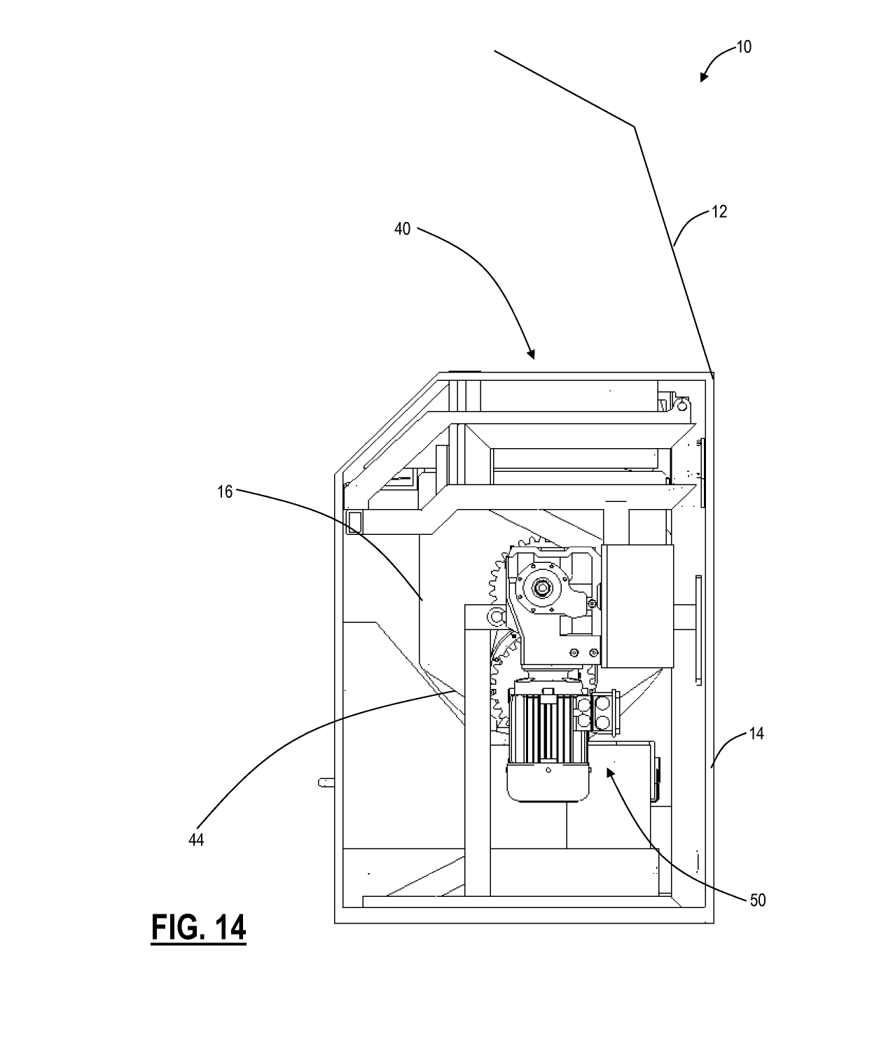

FIG. 7 is a front perspective view of the frame 14 of the biomedical waste sterilizing system 10. FIG. 8 is a rear perspective view of the biomedical waste sterilizing system 10 with all components included and with the cover 12 closed. FIG. 9 is a cross-sectional view of the biomedical waste sterilizing system 10 showing an X-Z plane view, illustrating a vessel 16 and chemical loading system 18. FIG. 10 is a cross-sectional view of the biomedical waste sterilizing system 10 showing an X-Y plane view. FIG. 11 is a cross-sectional view of the biomedical waste sterilizing system 10 showing a Y-Z plane view. FIG. 12 is a perspective view of the vessel 16 and a drawer 20 of the biomedical waste sterilizing system 10. FIG. 13 is a top view of the biomedical waste sterilizing system 10 with the cover 12 removed. 16 FIG. 14 is a side view of the biomedical waste sterilizing system 10 with the cover 12 in an open position.

Again, in an exemplary aspect, the biomedical waste sterilizing system 10 is designed to minimize capital costs. The biomedical waste sterilizing system 10 includes the frame 14 which is a minimal support structure for all other components in the biomedical waste sterilizing system 10. In an exemplary embodiment, illustrated in FIG. 7, the frame 14 is substantially rectangular or square. The frame 14 can include a top side 22, a bottom side 24, a front side 26, a back side 28, a left side 30, and a right side 32. Each of the sides 22, 24, 26, 28, 30, 32 can be substantially open, i.e., the frame 14 and the biomedical waste sterilizing system 10 does not include a housing which encloses all of the components. The frame 14 can be a plurality of interconnect members, railings, rails, or the like (collectively, members 34) composed of metal, plastic, or any other suitable rigid composition, collectively forming a rigid support structure for the other components of the biomedical waste sterilizing system 10. The members 34 at various meeting points for the sides 22, 24, 26, 28, 30, 32, and the 22, 24, 26, 28, 30, 32 are open.

In an exemplary aspect, the biomedical waste sterilizing system 10 utilizes liquid ozone and thus does not utilize harsh chemicals which are especially dangerous to humans based on touch or smell. Thus, the sides 22, 24, 26, 28, 30, 32 do not require full encasement of the components in a sealed housing for safety. Rather, the frame 14's primary function is for support of the biomedical waste sterilizing system 10 components. Thus, elimination of a housing is one exemplary aspect of cost reduction with the biomedical waste sterilizing system 10. The front side 26 can include a tilted portion 36 which can include an opening 38 for a user interface (not shown). The user interface can include, without limitation, buttons, a touch screen, a display, or any other input/output mechanism for user monitoring and control of the biomedical waste sterilizing system 10.

The vessel 16 is configured to receive biomedical waste, treat/sterilize the biomedical waste, and transfer the treated biomedical waste to the drawer 20. In an exemplary embodiment, illustrated in FIG. 12, the vessel 16 includes an opening 40 at a top of the vessel 16 configured to receive biomedical waste when the cover 12 is opened. The opening 40 provides access to a treatment chamber 42 where the biomedical waste is treated. Specifically, the biomedical waste sterilizing system 10 is a top loading system and top treatment system where the vessel 16 is positioned substantially in a vertical position for receiving and treating the biomedical waste.

Note, the vessel 16 is open at its top with the opening 40. The cover 12 is configured to cover the opening 40 during treatment cycles and during non-use of the biomedical waste sterilizing system 10. The cover 12 is rotatable about a top, rear member 34 on the frame 14. That is, the cover 12 is not physically attached to the vessel 16, but rather the frame 14.

The treatment chamber 42 is substantially square or rectangular at the opening 40 extending downward to a bottom portion 44 that includes angled sides on a front and back side of the vessel 16. That is, the bottom portion 44 tapers downward to an opposite end of the vessel 16 from the opening, i.e., a bottom end of the vessel 16. Cutting teeth 46 are located in the bottom portion 44 of the treatment chamber 42. The cutting teeth 46 can be disposed of on a cylinder 48 that is rotatably disposed to left and right sides of the vessel 16. Specifically, the cylinder 48 is connected to a gear system 50 next to the vessel 16 and disposed to/within the frame 14.

The gear system 50 is configured to rotate the cylinder 48 which correspondingly rotates the cutting teeth 46 to treat biomedical waste in the treatment chamber 42. Note, the gear system 50 can rotate the cylinder 48 clockwise, counter-clockwise, or a combination of both. Also, the gear system 50 can rotate the cylinder 48 at various speeds. The gear system 50 performs the rotation as part of a treatment cycle to shred and treat the biomedical waste with water and treatment chemicals in the water.

The cylinder 48 connects the gear system 50 through openings 52 on the left and right sides of the vessel 16. The left and right sides of the vessel 16 also include notches 54. Specifically, the vessel 16 is configured to tilt between two positions—an upright position for loading and treatment and a titled position for transferring treated waste to the drawer 20. Again, the biomedical waste sterilizing system 10 is a top loading and then tilt emptying system. In this manner, the biomedical waste sterilizing system 10 minimizes the tilting positions of the vessel 16, both reducing complexity and cost of the biomedical waste sterilizing system 10.

A pressing system 56 includes members 58 connected to the vessel 16 that collectively are configured provide a pressing mechanism once biomedical waste is introduced into the treatment chamber 42. The pressing mechanism is configured to press over the vessel 16 and press down. The pressing system 56 can be used to position biomedical waste for treatment in the treatment chamber 42 as well as for compacting already treated biomedical waste.

A gear system 60 is configured to tilt or rotate the vessel 16 about the notches 54 after treatment is complete, allowing the transfer of treated waste to the drawer 20. Note, this tilting operation is performed with lower cost and complexity than electric or mechanical actuators. In an exemplary embodiment, the vessel 16 can be tilted approximately 135 degrees from an upright position to transfer treated waste to the drawer 20. The drawer 20 is configured to slide open and closed for removal of the treated waste.

For treatment, a water system 62 is configured to provide water to the treatment chamber 42. Note, the water can be hot, cold, warm, etc. In addition to the water, the chemical loading system 18 is configured to accept chemicals for inclusion with the water to treat, sterilize, and shred the biomedical waste.

In an exemplary embodiment, the biomedical waste sterilizing system 10 utilizes liquid ozone for the chemicals to treat the biomedical waste. Note, liquid ozone is used as opposed to Hydrogen Peroxide, Peracetic acid or chlorine based compounds. Also, the liquid ozone does not require an on-site ozone generator with the biomedical waste sterilizing system 10. Again, this reduces cost and complexity of operation of the biomedical waste sterilizing system 10.

In the upper atmosphere, ozone provides protection from harmful ultraviolet radiation. Closer to Earth, ozone purities and sanitizes the air, water, etc. Ozone has shown remarkable antibacterial, anti-viral, and anti-fungal properties. Ozone is a form of oxygen, including three molecules (O₃). However, ozone is very unstable and returns to O₂ oxygen within minutes, under normal atmospheric conditions. For this reason, all conventional ozone treatment approaches use an on-site generator of ozone for use immediately for treatment. Again, this significantly increases the cost of the system, requiring an on-site ozone generator.

Oxygen radicals are powerful oxidizing agents able to induce oxidation-reduction reactions on various molecules including metals, nitrogen oxides, carbon, ammonia, sulfides and the like. Oxygen radicals are effective in the disinfection/sanitization of pathogens including viruses, bacteria, fungi as well as prions. Oxygen radicals also promote the oxidation of carbon-carbon double bonds (C═C) and ortho bridges (C═O═C). These types of bonds are found in many biologic molecules and other types of organic compounds, most notably pharmaceuticals, all of which can be sterilized via the biomedical waste sterilization system 10

Oxygen radicals are very strong oxidants that are used to break up the molecular structure of pharmaceuticals. In the present disclosure, oxygen radicals delivered by (product name) is used to treat pharmaceutical waste (hazardous and non-hazardous) as well as chemical and biomedical waste. Advantageously, the liquid ozone avoids on-site generation of gaseous ozone which is both costly and dangerous. Note, on-site ozone generation systems are sealed, protected, monitored and alarmed to prevent leaking. This adds cost, additional structure, and the like. The liquid ozone is concentrated, in water, which is safer and lower cost (removing the requirement for on-site ozone generation equipment).

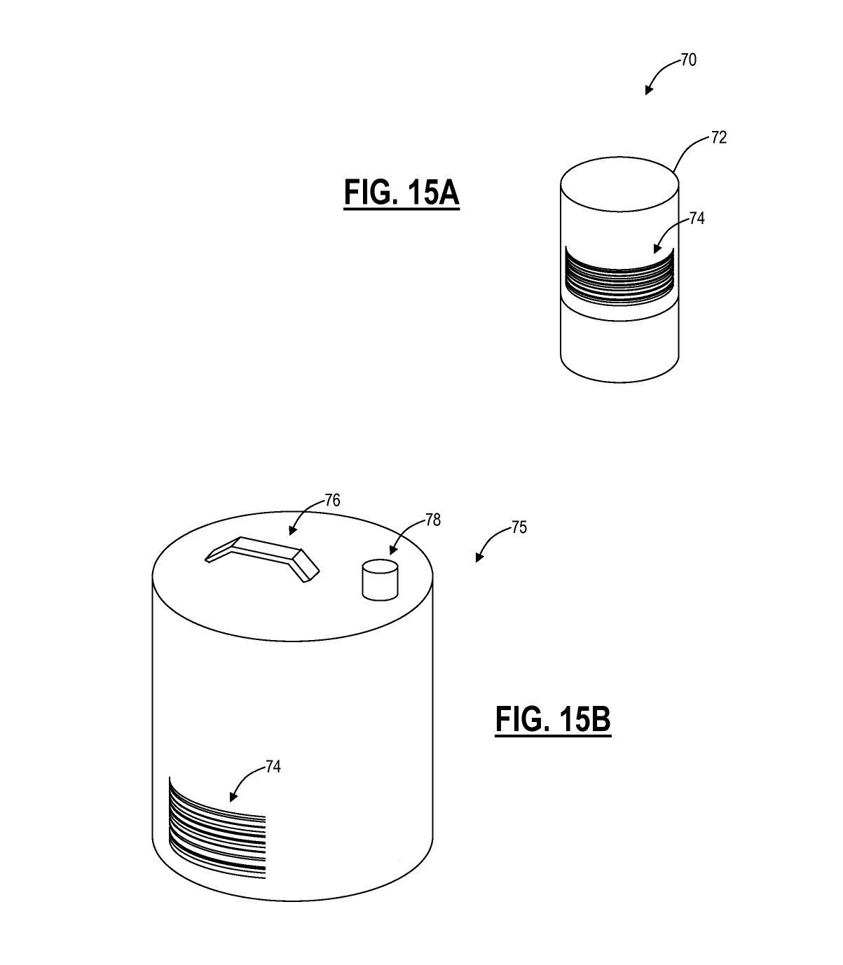

Referring to FIG. 15A, in an exemplary embodiment, a perspective diagram illustrates a liquid ozone capsule 70 for use in the biomedical waste sterilizing system 10. Specifically, the liquid ozone capsule 70 is placed in the chemical loading system 18. The liquid ozone capsule 70 can also be referred to a pod, tablet, etc. Also, liquid ozone can be referred to as aqueous ozone, denoting that water is the liquid in which the ozone is infused. The liquid ozone capsule 70 includes ozone infused into water.

The liquid ozone capsule 70 includes a higher disinfection range, for the management of viruses, bacteria, molds and biofilm than gaseous ozone or other types of chemicals. Specifically, the liquid ozone capsule 70 turns ordinary tap water into the world's most effective chemical-free commercial cleaner and sanitizer by infusing it with ozone. The liquid ozone capsule 70 eliminates germs, odors, stains, mold, mildew and other contaminants in the treatment chamber before changing back to water and oxygen. That is, the liquid ozone capsule 70 turns leaves no residues behind.

The liquid ozone capsule 70 includes a dissolvable membrane 72 that encases liquid ozone. The dissolvable membrane 72 dissolves once the liquid ozone capsule 70 enters the treatment chamber along with water. Additionally, one exemplary aspect of the biomedical waste sterilizing system 10 is to reduce both capital and operating costs. For example, the biomedical waste sterilizing system 10 can be operated by small or medium size medical facilities on a pay-as-you-go or use basis. This enables the small or medium size medical facilities to deploy the biomedical waste sterilizing system 10 without up-front costs, solely paying for use on a fixed or variable basis.

Note, by infusing the ozone in liquid and encasing the liquid ozone in the dissolvable membrane 72, the shelf-life of the liquid ozone is significantly expanded over conventional ozone (which is in minutes). The shelf-life of the liquid ozone capsule 70 can be in months, thereby allowing distribution of the liquid ozone capsule 70 rather than an on-site generation.

To support the pay-as-you-go or use basis, the liquid ozone capsule 70 includes an identifier 74 on the membrane 72. Operationally, the biomedical waste sterilizing system 10 can be configured to only accept the liquid ozone capsule 70 based on a verification of the identifier 74. For example, the identifier 74 can include a barcode (e.g., 2D code, Quick Response (QR) code, etc.), Radio Frequency Identifier (RFID), or any other type of identifier that can be quickly scanned for verification by the biomedical waste sterilizing system 10. This is a key enabler to the pay-as-you-go or use basis. That is, an operator can receive the biomedical waste sterilizing system 10 for little or no cost, but there has to be verifiable mechanisms to show how much use is performed for billing. The identifier 74 is also used to check the expiration date of the liquid ozone capsule 70.

The identifier 74 can be input into the biomedical waste sterilizing system 10, such as via a scanner or reader, and verified. The identifier 74 can be a single use verification. Once verified, the liquid ozone capsule 70 can be placed in the chemical loading system 18 after biomedical waste is loaded into the treatment chamber 42.

To create and use the liquid ozone capsule 70, oxygen (O₂) is turned into ozone (O₃) and infused into water, e.g., tap water. This step can be performed off-site, relative to a location of the biomedical waste sterilizing system 10. The liquid ozone capsule 70, with appropriate identifier 74, is provided to an operator of the biomedical waste sterilizing system 10. The biomedical waste sterilizing system 10 verifies the identifier 74 to accept the liquid ozone capsule 70.

The liquid ozone capsule 70 generates reactive oxygen species (also known as ozone, singlet oxygen, hydroxyl, peroxide and others.) These oxygen species have a high energy about them that attracts other ions on the surface of pathogens and organics. From this attraction oxidation occurs which destroys the DNA of the pathogen and oxidizes others.

In the treatment chamber 42 and with water, the membrane 72 dissolves, releasing the liquid ozone in the treatment chamber 42. In addition to the shredding and mixing of the biomedical waste with the cutting teeth 46, the extra atom from the ozone is fatally attracted to pathogens and contaminants in the biomedical waste. Harmless to people, the extra oxygen atom actively detaches and attacks and destroys bacteria, viruses, and contaminates. When complete, the ozone turns back into oxygen leaving only water and oxygen remaining with the treated waste after sanitizing has taken place.

Referring to FIG. 15B, a perspective diagram illustrates a liquid ozone drum 75 for use in the biomedical waste sterilizing system 10. The liquid ozone capsule 70 provides a specific amount of liquid ozone for a treatment cycle whereas the liquid ozone drum 75 can provide a variable amount of liquid ozone for the treatment cycle, based on feedback. For example, some waste may require much more ozone than other waste. Thus, the liquid ozone drum 75 supports on-demand distribution of ozone for a treatment cycle. Enough liquid ozone is used until it is determined, based on feedback from ORP/pH sensors that the waste is disinfected.

The liquid ozone drum 75 can be any size, such as 5 gallons or the like. The liquid ozone drum 75 includes the identifier 74, which similar to the liquid ozone capsule 70, can provide information related to expiration date, volume, billing, etc. The liquid ozone drum 75 can include a handle 76 for transport and an opening point 78. The biomedical waste sterilizing system 10 can include a cavity/holding mechanism to receive the liquid ozone drum 75. In an exemplary embodiment, the opening point 78 is a cap or lid that is removable and configured to interface to the chemical loading system 18. In another exemplary embodiment, to avoid exposure to the liquid ozone, the chemical loading system 18 can include a sharp pin or the like which pierces the opening point 78 when the liquid ozone drum 75 is loaded in the cavity.

When the liquid ozone drum 75 is empty, it is not dangerous due to the liquid ozone. The liquid ozone drum 75 can be recycled or actually disposed of through a treatment cycle with the biomedical waste sterilizing system 10.

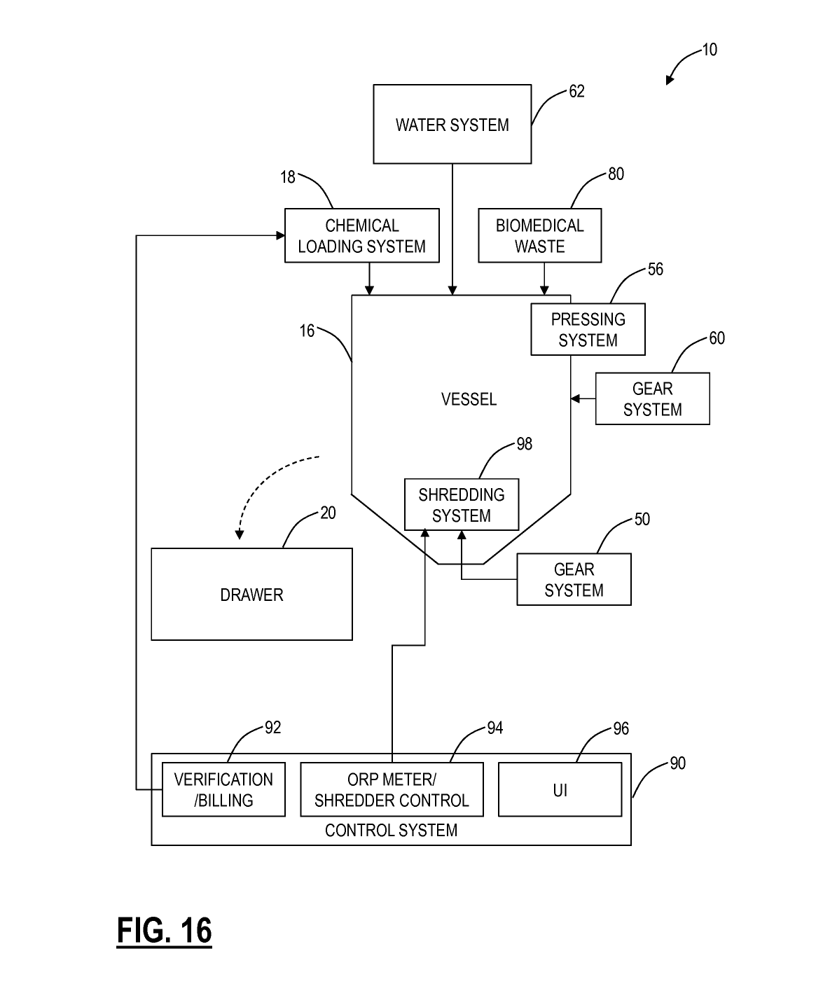

Referring to FIG. 16, in an exemplary embodiment, a block diagram illustrates the functional operation of the biomedical waste sterilizing system 10. FIG. 16 provides a functional view of the various components in the biomedical waste sterilizing system 10, and, for illustration purposes, is described relative to an exemplary operation of the biomedical waste sterilizing system 10.

Biomedical waste 80 is introduced into the treatment chamber 42 via the opening 40. Again, the biomedical waste 80 includes, without limitation, discarded blood, unwanted microbiological cultures and stocks, identifiable body parts, other human or animal tissue, used bandages and dressings, discarded gloves, other medical supplies that may have been in contact with blood and body fluids, laboratory waste that exhibits the characteristics described above, pharmaceutical compounds such as expired or discarded drugs, etc. The biomedical waste 80 can be pressed, via the pressing system 56, and water can be introduced into the treatment chamber 42 via the water system 62. Note, despite being described as biomedical waste 80, the biomedical waste 80 also includes pharmaceutical waste.

Operations of the biomedical waste sterilizing system 10 can be monitored and controlled via a control system 90. The control system 90 can include a verification/billing component 92, an Oxidation-Reduction Potential (ORP) meter/shredder control component 94, and a User Interface (UI) 96.

The control system 90 may include one or more generic or specialized processors (“one or more processors”) such as microprocessors, digital signal processors, customized processors, and field programmable gate arrays (FPGAs) and unique stored program instructions (including both software and firmware) that control the one or more processors to implement, in conjunction with certain non-processor circuits, some, most, or all of the functions of the control system 90. Alternatively, some or all functions of the control system 90 may be implemented by a state machine that has no stored program instructions, or in one or more application-specific integrated circuits (ASICs), in which each function or some combinations of certain of the functions are implemented as custom logic. Of course, a combination of the aforementioned approaches may be used. Moreover, some exemplary embodiments of the control system 90 may be implemented as a non-transitory computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), Flash memory, and the like. When stored in the non-transitory computer readable medium, software can include instructions executable by a processor that, in response to such execution, cause a processor or any other circuitry to perform a set of operations, steps, methods, processes, algorithms, etc. for the control system 90.

The liquid ozone capsule 70 can be verified via the verification/billing component 92 which serves two functions—1) ensures the usage and billing of the waste sterilizing system 10 with the liquid ozone capsule 70 and 2) ensures the liquid ozone capsule 70 is a proper dosage and expiration date. That is, an operator can interact with the UI 96 to determine a type of treatment for whatever type of waste is associated with the biomedical waste 80. The control system 90 can be configured to ensure the liquid ozone capsule 70 is a proper dose (based on the identifier 74) based on the UI 96 input.

Once verified and approved, the operator can insert the liquid ozone capsule 70 into the chemical loading system 18. With the water and the liquid ozone capsule 70 in the treatment chamber 42, the control system 90 can start a treatment cycle which includes controlling a shredding system 98 (e.g. the cutting teeth 46 and the cylinder 48 controlled by the gear system 50). The control system 90 controls the treatment cycle via the ORP meter/shredder control component 94.

The ORP meter/shredder control component 94 is configured to monitor an ORP value of the water in the treatment chamber 42 during the treatment cycle. ORP is a measure of the cleanliness of the water and its ability to break down contaminants. ORP can have a range, e.g., of −2,000 to +2,000 mV, but since ozone is an oxidizer, only positive ORP levels are of concern.

An ORP meter or sensor is located in the treatment chamber 42 and configured to measure the dissolved oxygen. More contaminants in the treatment chamber 42 result in less dissolved oxygen because the organics are consuming the oxygen and therefore, the lower the ORP level. For effective treatment of the biomedical waste 80, ORP levels above 600 mV are required. The ORP meter/shredder control component 94 can be configured to control the shredding system 98 based on feedback from the ORP meter or sensor. In another exemplary embodiment, the ORP meter or sensor can be replaced or augmented by a pH meter which continually measures a pH level of the water in the treatment chamber 42. In a further exemplary embodiment, both ORP and PH levels can be determined and monitored. In yet another exemplary embodiment, a weight or mass can be determined for input biomedical waste 80, and measured during/after treatment.

The on-demand distribution of liquid ozone includes provides more or less ozone, such as from the liquid ozone drum 75 based on the feedback, from the ORP meter or sensor, from the pH meter, from load cells for weight, and the like.

Once complete, the vessel 16 is tilted by the gear system 60 to transfer the treated biomedical waste 80 to the drawer 20 for disposal.

In an exemplary embodiment, a biomedical waste sterilization system 10 includes a vessel 16 including a treatment chamber 42, wherein the vessel 16 is disposed in an open frame 14 for support thereof and the vessel 16 includes an opening 40 on a top side configured to receive biomedical waste 80 and a shredding system 98 to treat the biomedical waste 80; and a chemical loading system 18 configured to receive liquid ozone, such as via the liquid ozone capsule 70, for introduction into the treatment chamber 42 prior to a treatment cycle for the biomedical waste 80. The vessel 16 can be configured to receive the biomedical waste 80 and to treat the biomedical waste 80 in a top loading position, and wherein the vessel 16 is further configured to tilt after the treatment cycle to remove treated biomedical waste 80.

The vessel 16 can be configured to tilt using a gear system 60 coupled to the vessel 16. The biomedical waste sterilization system 10 can further include a cover 12 rotatably connected to a member 34 of the frame 14 and configured to cover the opening 40 in the vessel 16 during the treatment cycle and non-use and to allow access to the opening 40 during loading of the biomedical waste 80. Optionally, the cover 12 is not physically attached or connected to the vessel 16. The shredding system 98 can include a cylinder 48 with a plurality of cutting teeth 46 disposed thereon and a gear system 50 configured to rotate the cylinder 48, and wherein the cylinder 48 is located in a bottom portion 44 of the vessel 16. The gear system 50 can be configured to rotate the cylinder 48 both clockwise and counter-clockwise at variable speeds, based on feedback from a control system 90 associated with the biomedical waste sterilization system 10.

The biomedical waste sterilization system 10 can further include a control system 90 configured to monitor and control operation of the biomedical waste sterilization system 10, the control system can include a verification/billing component 92, a shredder control component 94, and a User Interface 96. The control system 90 can be configured to verify and track liquid ozone use through an associated identifier 74 as well as ensure proper expiration dates for the liquid ozone. The control system 90 is configured to operate the shredder control component 94 based on feedback from one of an Oxidation-Reduction Potential (ORP) meter or sensor and a pH meter in the treatment chamber 42.

The liquid ozone can include a liquid ozone capsule 70 that is created off-site such that the biomedical waste sterilization system 10 does not include an on-site ozone generator. The liquid ozone capsule 70 can include a dissolvable membrane 72 encasing a solution of ozone infused into water and an identifier 74 on the dissolvable membrane 72 configured to be scanned or read by the biomedical waste sterilization system 10 prior to use.

In another exemplary embodiment, the chemical loading system 18 can include the drum 75 or other container housing liquid ozone. This exemplary embodiment does not rely on a capsule or the like, i.e., a user does not load liquid ozone in this exemplary embodiment for each cycle. The container housing liquid ozone is configured to release a variable amount of liquid ozone into the vessel 16 based on measurements during a treatment cycle, i.e., ORP and/or pH measurements. Here, the shredder control component 94 is configured to release liquid ozone from the chemical loading system 18 based on feedback from the ORP meter and/or pH sensor.

The biomedical waste 80 may include a significant amount of waste that needs to be treated with the liquid ozone or a small amount. The feedback from the ORP meter and/or pH sensor can be used to adequately address the biomedical waste 80 during the treatment cycle.

Note, in another exemplary embodiment, the chemical loading system 18 can include both a capsule-based system and a drum or other container housing liquid ozone. The capsule 70 can provide a baseline amount of liquid ozone for treatment. The drum or other container can be used for additional liquid ozone as needed to adequately address the biomedical waste 80 during a treatment cycle.

Also, the verification/billing component 92 can work with the drum or other container for the liquid ozone, monitoring a usage amount and billing based on an amount of liquid ozone used. Again, chemical loading system 18 contemplates liquid ozone to avoid having to generate ozone gas on-site with the biomedical waste sterilizing system 10. The verification/billing component 92 can work with the identifier 74, based on metering with the drum, and the like.

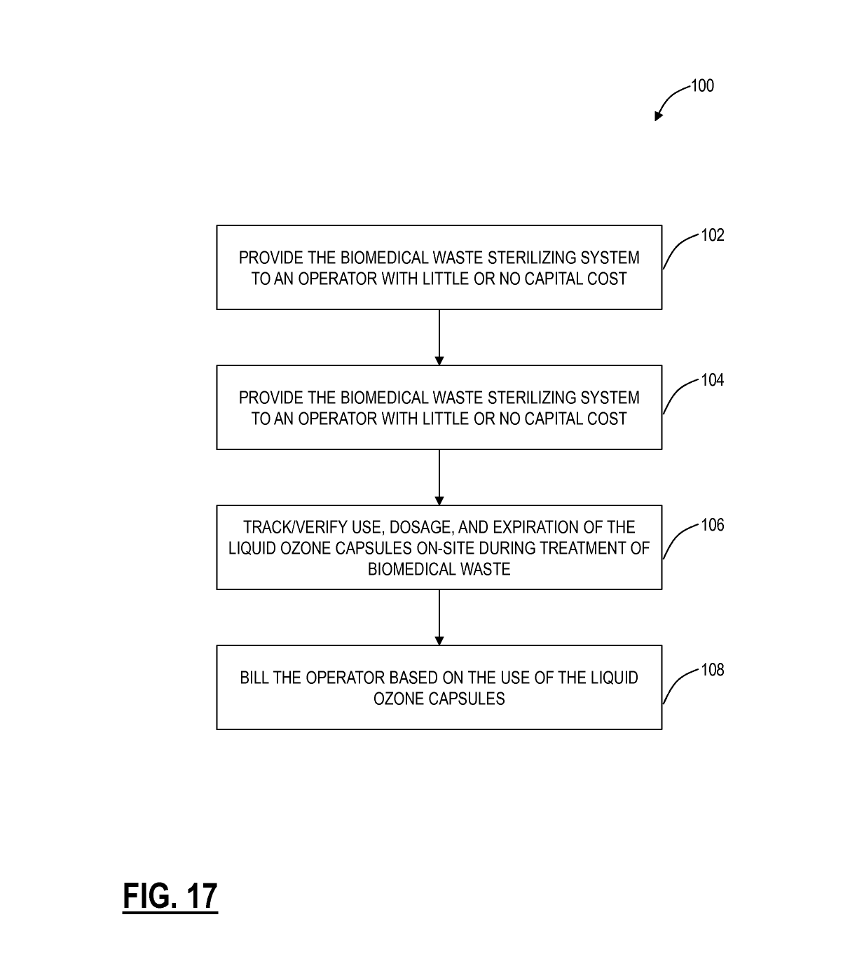

Referring to FIG. 17, in an exemplary embodiment, a flowchart illustrates a deployment and operational process 100 of the biomedical waste sterilizing system 10. The deployment and operational process 100 provides a pay-as-you-go or use solution using the biomedical waste sterilizing system 10. This advantageously allows small, medium, or even large size medical facilities opportunities to use the biomedical waste sterilizing system 10 without excessive capital or operating costs.

The deployment and operational process 100 includes providing the biomedical waste sterilizing system 10 to an operator with little or no capital cost (step 102), providing the operator one or more liquid ozone capsules 70 (step 104), tracking/verifying use, dosage, and expiration of the liquid ozone capsules 70 on-site during treatment of biomedical waste 80 (step 106), and billing the operator based on the use of the liquid ozone capsules 70 (step 108).

Note, the design and operation of the biomedical waste sterilizing system 10 is optimized to reduce costs, both operational and capital. In an exemplary embodiment, deployment of the biomedical waste sterilizing system 10 can utilize the deployment and operational process 100 allowing a fixed cost for the operator.

The inherent ability for liquid ozone to destroy viruses and bacteria is dependent on two main variables: initial concentration of the ozone in the liquid (ppm or parts per million) and contact or dwell time. The sanitizing ability of the dissolved ozone increases as either or both variable is increased. The following tables measure the power of aqueous ozone and time required to destroy bacteria and viruses at a strength of 2 ppm or greater:

| | Reduction (%) | Dwell Time (Secs.) |
|---|---|---|
| Bacteria | | |
| Escherichia coli | 99.99 | 5-13 |
| Listeria monocytogenes | 99.999 | 3-11 |
| Salmonella typhimurium | 99.99 | 11-13 |
| Streptococcus faecalis | 99.999 | 23-26 |
| Legionella pneumophila | 99.99 | 9-33 |
| Bacillus cereus | 99.9999 | 33 |
| Viruses | | |
| Bacteriophage F2 | 99.999 | 2-19 |
| Norovirus | 99.9 | 2 |
| Hepatitus A | 99.9 | 1 |
| Poliovirus type 1 | 99.9 | 5 |
| Rotavirus | 99.99 | 63-126 |

The following list provides typical dosage and dwell times
Bacillus Bacteria: Destroyed by 0.2 mg/l within 30 seconds
Bacillus Anthracis: Causes anthrax in sheep, cattle and pigs. A human pathogen. Ozone susceptible.
Clostridium Bacteria: Ozone-Susceptible.
Clostridium Botulinum Spores: Its toxin paralyzes the central nervous system, being a poison multiplying in food and meals. 0.4 to 0.5 mg/l.
Echo Virus 29: This virus most sensitive to ozone. After a contact time of 1 Minute at 1 mg/l of ozone,
   99.999% killed.
Escherichia Coli Bacteria (from feces): Destroyed by 0.2 mg/l within 30 seconds.
Encephalomyocarditis Virus: Destroyed to zero level in less than 30 seconds with 0.1 to 0.8 mg/l.
Enterovirus Virus: Destroyed to zero level in less than 30 seconds with 0.1 to 0.8 mg/l.
GDVII Virus: Destroyed to zero level in less than 30 seconds with 0.1 to 0.8 mg/l.
Herpes Virus: Destroyed to zero level in less than 30 seconds with 0.1 to 0.8 mg/l.
Influenza Virus: 0.4 to 0.5 mg/l.
Poliomyelitis Virus: Kill of 99.999% with 0.3 to 0.4 mg/l
Proteus Bacteria: Very Susceptible.
Pseudomonal Bacteria: Very Susceptible.
Rhabdovirus Virus: Destroyed to zero level in less than 30 seconds.
Salmonella Bacteria: Very Susceptible.
Stomatitis Virus: Destroyed to zero level in less than 30 seconds with 0.1 to 0.8 mg/l.
Streptococcus Bacteria: Destroyed by 0.2 mg/l within 30 seconds
Aspergillus Niger (black Mount): Destroyed by 1.5 to 2 mg/l.
Diphtheria Pathogen: Destroyed by 1.5 to 2 mg/l.
Eberth Bacillus (Typhus abdominalis): Destroyed by 1.5 to 2 mg/l.
Klebs-Loffl er Virus: Destroyed by 1.5 to 2 mg/l.
Staphylococci: Destroyed by 1.5 to 2 mg/l.

The following table illustrates an exemplary list of common organisms killed by the liquid ozone capsule 70:

| | | |
|---|---|---|
| Pseudomonas putida | Escherichia coli | Cysts |
| Salmonella choleraesuis | Flavorbacterium SP A-3 | Cryptosporidium parvum |
| Salmonella enteritidis | Leptospira canicola | Giardia lamblia |
| Salmonella typhimurium | Listeria | Giardia muris |
| Salmonella typhosa | Micrococcus candidus | Algae |
| Salmonella paratyphi | Micrococcus caseolyticus | Chlorella vulgaris |
| | KM-15 | |
| Sarcina lutea | Micrococcus spharaeroides | Thamnidium |
| Seratia marcescens | Mycobacterium leprae | Trichoderma viride |
| Shigella dysenteriae | Mycobacterium tuberculosis | Verticillium albo-atrum |
| Shigella flexnaria | Neisseria catarrhalis | Verticillium dahliae |
| Shigella paradysenteriae | Phytomonas tumefaciens | Virus |
| Spirllum rubrum | Proteus vulgaris | Adenovirus (type 7a) |
| Staphylococcus albus | Pseudomonas aeruginosa | Bacteriophage (E. coli) |
| Staphylococcus aureus | Pseudomonas | Coxackie A9, B3, & B5 |
| Streptococcus ‘C’ | fluorscens (bioflims) | Cryptosporidium |
| Streptococcus faecalis | Fungus & Mold Spores | Echovirus 1, 5, 12, &29 |
| Streptococcus hemolyticus | Aspergillus candidus | Encephalomyocarditis |
| Streptococcus lactis | Aspergillus flavus (yellowish- | Hepatitis A |
| | green) | |
| Streptococcus salivarius | Aspergillus glaucus (bluish- | GD V11 Virus |
| | green) | |
| Streptococcus viridans | Aspergillus niger (black) | Onfectious hepatitis |
| Torula rubra | Aspergillus terreus, saitoi & | Influenza |
| | oryzac | |
| Vibrio alginolyticus & | Botrytis allii | Legionella pneumophila |
| angwillarum | | |
| Vibrio clolarae | Colletotrichum lagenarium | Polio virus (Poliomyelitus) 1, |
| | | 2 & 3 |
| Vibrio comma | Fusarium oxysporum | Rotavirus |
| Virrio ichthyodermis NC-407 | Grotrichum | Tobacco mosaic |
| V. parahaemolyticus | Mucor recomosus A & B | Vesicular Stomatitis |
| | (white-gray) | |
| Bacteria | Mucor piriformis | Protozoa |
| Achromobacter butyri NCI- | Oospora lactis (white) | Paramecium |
| 9404 | | |
| Aeromonas harveyi NC-2 | Penicillium cyclopium | Nematode eggs |
| Aeromonas salmonicida NC- | P. chrysogenum & citrinum | Chlorella vulgaris (Algae) |
| 1102 | | |
| Bacillus anthracis | Penicillium digitatum (olive) | All Pathogenic and Non- |
| | | pathogenic forms of Protozoa |
| Bacillus cereus | Penicillium glaucum | Fungal Pathogens |
| B. coagulans | Penicillium expansum (olive) | Alternaria solani |
| Bacillus globigii | Penicillium egyptiacum | Botrytis cinerea |
| Bacillus licheniformis | Penicillium roqueforti (green) | Fusarium oxysporum |
| Bacillus megatherium sp. | Rhizopus nigricans (black) | Monilinia fruiticola |
| Bacillus paratyphosus | Rhizopus stolonifer | Monilinia laxa |
| B. prodigiosus | Yeast | Pythium ultimum |
| Bacillus subtilis | Baker's yeast | Phytophthora erythroseptica |
| B. stearothermophilus | Candida albicans-all forms | Phytophthora parasitica |
| Clostridium botulinum | Common yeast cake | Rhizoctonia solani |
| C. sporogenes | saccharomyces cerevisiae | Rhizopus stolonifera |
| Clostridium tetoni | saccharomyces ellipsoideus | Sclerotium rolfsii |
| Cryptosporidium | saccharomyces sp. | Sclerotinia sclerotiorum |
| Coliphage | | |
| Corynebacterium diphthriae | | |
| Eberthella typhosa | | |
| Endamoeba histolica | | |

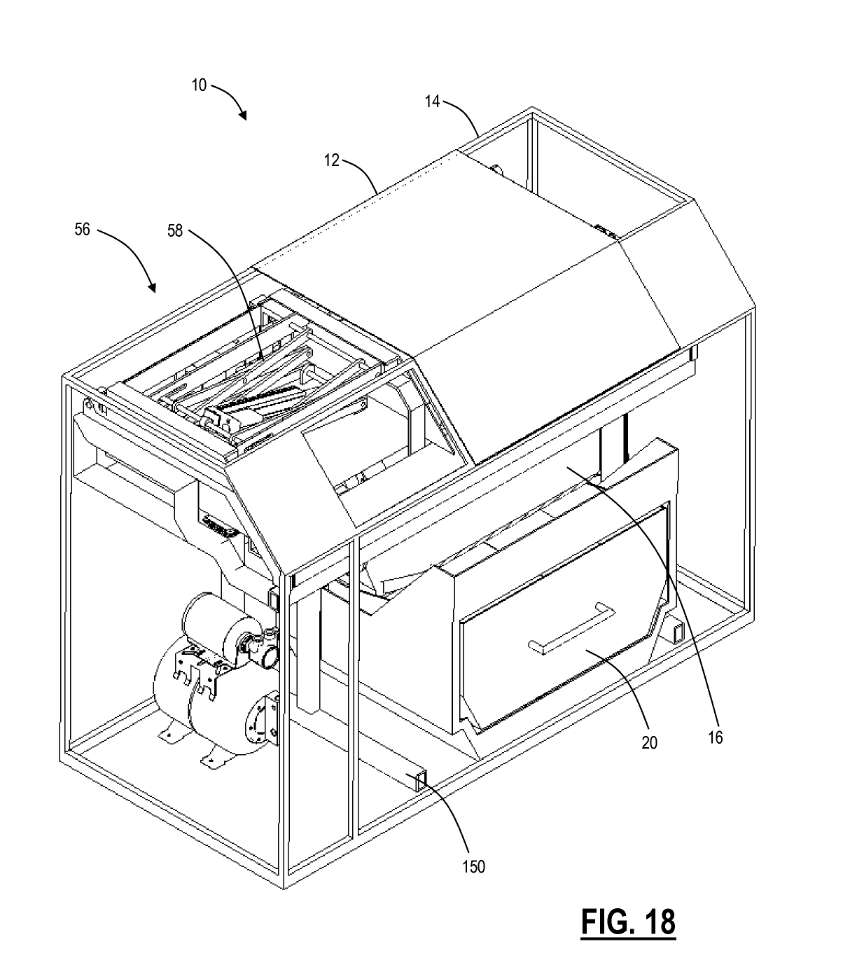

Referring to FIG. 18, in an exemplary embodiment, a front perspective view illustrates the biomedical waste sterilizing system 10 with load cells 150 configured to determine weight of the biomedical waste 80. The load cells 150 are located a feet or other support structures associated with the biomedical waste sterilizing system 10. The load cells 150 are transducers that create an electrical signal whose magnitude is directly proportional to the force being measured. The load cells 150 are initialized to determine an unloaded weight, and then configured to measure the weight of the biomedical waste 80. Note, the feedback during the treatment cycle can include the varying weight as one monitored variable, in addition to ORP, pH, etc.

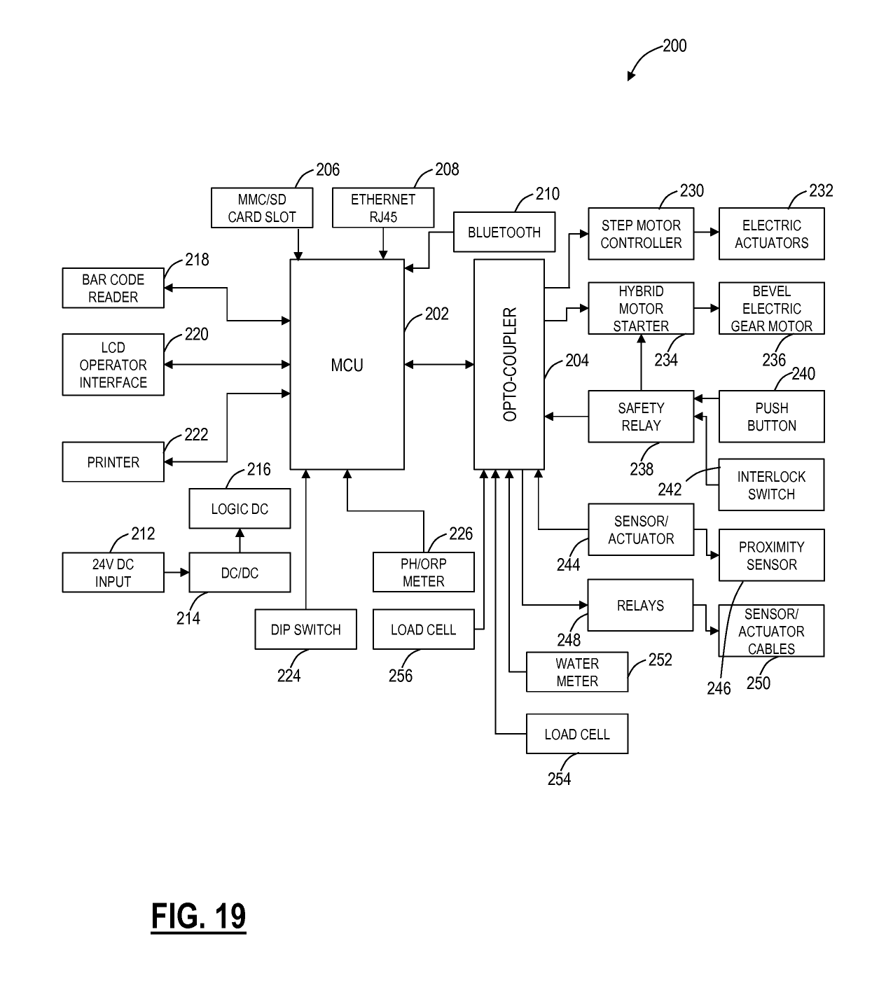

Referring to FIG. 19, in an exemplary embodiment, a block diagram illustrates an integrated circuit (IC)-based control system 200 for use with the biomedical waste sterilizing system 10. To control costs and provide flexibility, various functions are integrated and implemented in the IC-based control system 200 as opposed to discrete, disparate systems. The IC-based control system 200 includes a microcontroller unit (MCU) 202 communicatively coupled to an opto-coupler 204.

The MCU 202 is accessible via a MMC/SD card slot 206, an Ethernet port 208, and via Bluetooth 210. For power input, the MCU 202 and the opto-coupler 204 support 24V DC 212 which can be rectified (DC/DC 214) to provide logic DC power 216. For user input or output, the MCU 202 is communicatively coupled to a bar code reader 218, a liquid crystal display (LCD) operator interface 220, and a printer 222. The bar code reader 218 is configured to scan the identifiers 74, and the LCD operator interface 220 can implement the UI 96.

The MCU 202 can include connections to a DIP switch 224 and a pH/ORP meter 226. The DIP switch 224 can be used for DMX addressing with the MCU 202. The pH/ORP meter 226 is a pH/ORP meter associated with the vessel 16. In an exemplary embodiment, the pH/ORP meter 226 provides both pH and ORP readings. With the pH/ORP meter 226, the MCU 202 can provide Proportional-Integral-Derivative (PID), Proportional-Integral (PI), Proportional (P), and/or on/off control of pH and/or ORP for one or multiple set points. Thus, the pH/ORP meter 226 and the MCU 202 enable feedback control of a treatment cycle to determine liquid ozone amount, treatment time, etc. The pH/ORP meter 226 can also monitor temperature as well.

The opto-coupler 204 is configured to interact electrically with the MCU 202 and to interact optically with other components in the IC-based control system 200. The opto-coupler 204 is connected to a step motor controller 230 which connects to electric actuators 232 associated with the biomedical waste sterilizing system 10. The step motor controller 230 provides motion control based on data for position, speed, acceleration, and pushing force. The electric actuators 232 include step or servo motors to move portions of the biomedical waste sterilizing system 10, such as the vessel 16.

The opto-coupler 204 connects to a hybrid motor starter 234 which connects to a bevel electric gear motor 236. The hybrid motor starter 234 and the bevel electric gear motor 236 are configured to move the cylinder 48. The opto-coupler 204 connects to a safety relay 238 which connects to a push bottom 240 and an interlock switch 242, each are configured to shut down the bevel electric gear motor 236 as appropriate for safety.

The opto-coupler 204 connects to a sensor/actuator 244 which connects to a proximity sensor 246 and to relays 248 which connect to sensor/actuator cables 250. Finally, the opto-coupler 204 connects to a water meter 252 and load cells 254, 256. The water meter 252 can be used to measure/monitor flows of water and/or liquid ozone into the vessel 16. The load cells 254, 256 are configured to provide weight measurements to determine a weight of the biomedical waste 80 in the vessel 16.

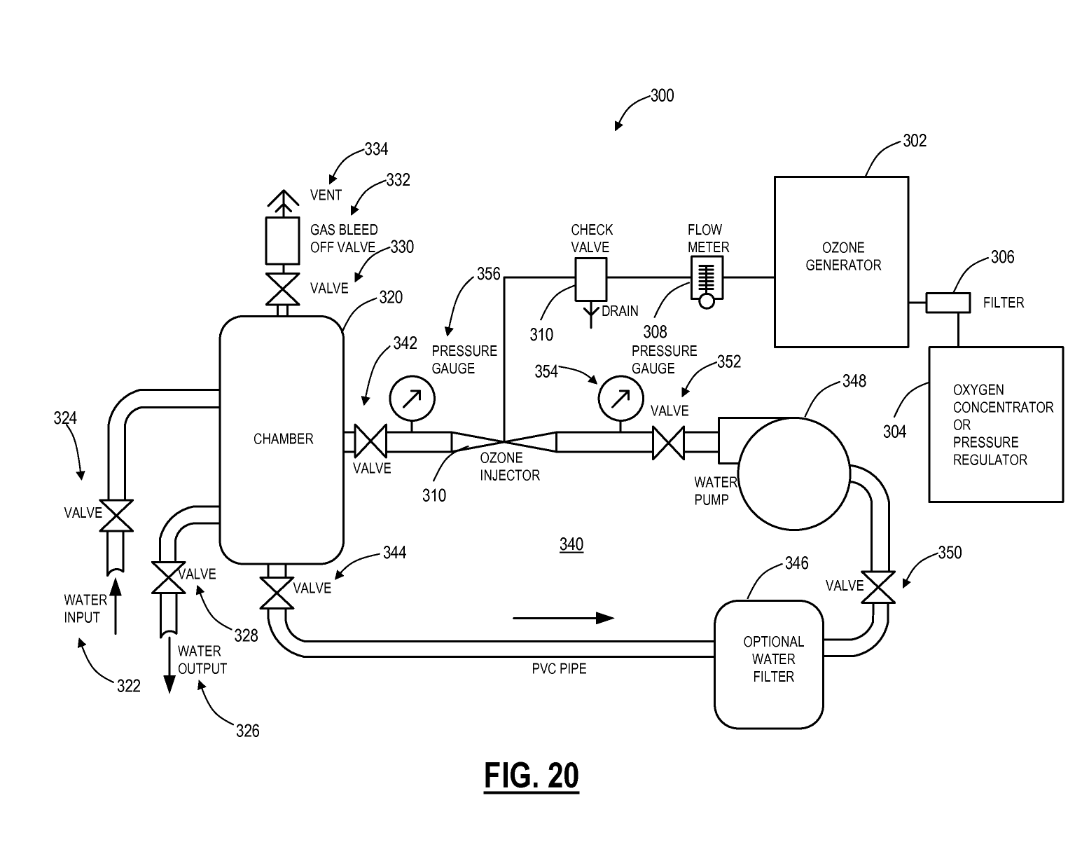

Referring to FIG. 20, in an exemplary embodiment, a block diagram functionally illustrates a biomedical waste sterilizing system 300 with ozone injection. Specifically, FIG. 20 illustrates a process diagram of the biomedical waste sterilizing system 300. Note, the physical implementation of the biomedical waste sterilizing system 300 can be similar to the biomedical waste sterilizing system 10 with an ozone generator 302 and the like included for ozone injection. The biomedical waste sterilizing system 300 is configured to inject ozone gas into water, using the ozone generator 302 which is connected to an oxygen regulator 304 (or pressure regulator) through a filter 306. The regulator 304 provides filtered oxygen to the ozone generator 302 which generates ozone. The generated ozone 302 is provides, through a flow meter 308 and check valve 310 to an ozone injector 312.

Biomedical and pharmaceutical waste is placed in a chamber 320 sterilizing systems and methods with ozone injection. In an exemplary aspect, the biomedical waste sterilizing system 300 is configured to inject the ozone gas into water to create a disinfectant to treat the waste. The ozone gas is not directly injected as gas into the chamber 320. After the waste is in the chamber 320, water is applied on the waste in the chamber 320. The water can be tap water or the like from a water input 322 and/or ozonated (ozone-infused) water from the ozone injector 310. The water input 322 connects to the chamber 320 via a valve 324. The chamber 320 also has a water output 326 via a valve 328. As described herein, the chamber 320 has similar components and functionality as the vessel 16, including blades to grind and shred the waste. The water and waste that is grinded in the chamber 320 can be referred to as slurry.

Within the chamber 320, the water is extracted, via filtering, from the slurry, i.e., the water is separated from the slurry. The chamber 320 includes a valve 330 along with a gas bleed off valve 332 for a vent 334 out of the chamber 320. There is a circulation loop 340 for the water and the ozone, and the circulation loop 340 connects to the chamber via valves 342, 344. The circulation loop 340 is configured to take the water from the chamber 320, to optionally filter the water with a filter 346 and to pump the water back into the chamber 320 with a water pump 348. Prior to the water being pumped back into the chamber 320, the water is infused/injected with ozone via the ozone injector 310. The circulation loop 340 also include valves 350, 352 and pressure gauges 354, 356.

The ozone injector 310 can be a differential pressure injectors with internal mixing vanes, such as a Venturi injector. In an exemplary embodiment, the circulation loop 340 can be a pipe, such as a PVC pipe. Water is circulated by running the water through a pipe and through the ozone injector 310 to force Ozone into the water to create the disinfecting effect. The water is dumped again on top of the waste to keep adding ozone into the water. The ozone in water attacks pathogens, and during this process, the concentration of ozone diminishes in the chamber 320. This is one objective of the circulation loop 340, to continually adding more ozone, as needed, during the circulation. Also, water has a certain saturation level for ozone. The circulation is used to overcome this saturation level and to avoid wasting the ozone. That is, the circulation loop 340 allows an efficient, but suitable amount of ozone for the chamber 320.

The waste is treated in the chamber 320 and with water flowing through the circulation loop 340 until it is disinfected. For example, a treatment cycle may be 5-15 minutes. Note, the circulation loop 340 can be controlled by the control system 90 and use the ORP/pH readings to directly control the amount of ozone injected into water for introduction into the chamber 320.

In the biomedical waste sterilizing system 300, ozone can be added into the water in various ways. Existing ozone in the water can be returned to the chamber 320 via the circulation loop 340. Ozone can be directly applied into water in the chamber 320 such as via the vent 334 and the valves 332, 330. Also, ozone can be injected in the circulation loop 340. Again, the circulation loop 340 uses the control system 90, such as a meter to test the quality of the water running through the circulation loop 340 to determine if it is been disinfected by measuring the ppm of ozone in water, pH, ORP, etc. The meter readings are used to control ozone infusion and when a treatment cycle is completed. When the disinfection is sufficient, the circulation loop 340 can be used to wash out the waste with the water.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims.

What is claimed is:

1. A biomedical and pharmaceutical waste sterilization system with an ozone injection system, comprising:
a vessel comprising a treatment chamber, wherein the vessel is disposed in an open frame for support thereof and the vessel comprises an opening on a top side configured to receive biomedical waste and a shredding system to treat the biomedical waste; and
a circulation loop configured to provide water and ozone injection into the water into the treatment chamber and to receive water from the treatment chamber during a treatment cycle for the biomedical waste.

2. The biomedical and pharmaceutical waste sterilization system of claim 1, wherein the circulation loop comprises:
an ozone generator coupled to an ozone injector coupled to the treatment chamber;
a water pump coupled to the ozone injector;
a return pipe from the treatment chamber into the water pump, wherein the circulation loop runs from the treatment chamber to the water pump to the ozone injector and back to the treatment chamber.

3. The biomedical and pharmaceutical waste sterilization system of claim 2, wherein the circulation loop further comprises a water filter between the water pump and the treatment chamber.

4. The biomedical and pharmaceutical waste sterilization system of claim 1, wherein ozone is provided to treat waste in the treatment chamber by one or more of injecting ozone into water through the circulation loop, recirculating already ozone infused water through the circulation loop, and injecting ozone gas into water before introducing the water into the treatment chamber.

5. The biomedical and pharmaceutical waste sterilization system of claim 4, wherein an amount of ozone provided for the treatment is determined by a control system based on feedback from one of an Oxidation-Reduction Potential (ORP) meter or sensor and a pH meter monitoring water from the treatment chamber.

6. The biomedical and pharmaceutical waste sterilization system of claim 1, wherein the vessel is configured to receive the biomedical waste and to treat the biomedical waste in a top loading position, and wherein the vessel is further configured to tilt after the treatment cycle to remove treated biomedical waste.

7. The biomedical and pharmaceutical waste sterilization system of claim 6, wherein the vessel is configured to tilt using a gear system coupled to the vessel.

8. The biomedical and pharmaceutical waste sterilization system of claim 1, further comprising:
a cover rotatably connected to a member of the frame and configured to cover the opening in the vessel during the treatment cycle and non-use and to allow access to the opening during loading of the biomedical waste.

9. The biomedical and pharmaceutical waste sterilization system of claim 8, wherein the cover is not physically attached or connected to the vessel.

10. The biomedical and pharmaceutical waste sterilization system of claim 1, wherein the shredding system comprises a cylinder with a plurality of cutting teeth disposed thereon and a gear system configured to rotate the cylinder, and wherein the cylinder is located in a bottom portion of the vessel.

11. The biomedical and pharmaceutical waste sterilization system of claim 10, wherein the gear system is configured to rotate the cylinder both clockwise and counter-clockwise at variable speeds, based on feedback from a control system associated with the biomedical waste sterilization system.

12. The biomedical and pharmaceutical waste sterilization system of claim 1, further comprising:
a control system configured to monitor and control operation of the biomedical waste sterilization system, the control system comprising a verification/billing component, a shredder control component, and a User Interface.

13. The biomedical and pharmaceutical waste sterilization system of claim 12, wherein the control system is configured to operate the shredder control component based on feedback from one of an Oxidation-Reduction Potential (ORP) meter or sensor and a pH meter.

14. A biomedical and pharmaceutical waste sterilization method with an ozone injection system, comprising:
providing a vessel comprising a treatment chamber, wherein the vessel is disposed in an open frame for support thereof and the vessel comprises an opening on a top side configured to receive biomedical waste and a shredding system to treat the biomedical waste; and
providing a circulation loop configured to provide water and ozone injection into the water into the treatment chamber and to receive water from the treatment chamber during a treatment cycle for the biomedical waste.

15. The biomedical and pharmaceutical waste sterilization method of claim 16, wherein the circulation loop comprises:
an ozone generator coupled to an ozone injector coupled to the treatment chamber;
a water pump coupled to the ozone injector;
a return pipe from the treatment chamber into the water pump, wherein the circulation loop runs from the treatment chamber to the water pump to the ozone injector and back to the treatment chamber.

16. The biomedical and pharmaceutical waste sterilization method of claim 15, wherein the circulation loop further comprises a water filter between the water pump and the treatment chamber.

17. The biomedical and pharmaceutical waste sterilization method of claim 15, wherein ozone is provided to treat waste in the treatment chamber by one or more of injecting ozone into water through the circulation loop, recirculating already ozone infused water through the circulation loop, and injecting ozone gas into water before introducing the water into the treatment chamber.

18. The biomedical and pharmaceutical waste sterilization method of claim 17, wherein an amount of ozone provided for the treatment is determined by a control system based on feedback from one of an Oxidation-Reduction Potential (ORP) meter or sensor and a pH meter monitoring water from the treatment chamber.

19. An apparatus, comprising:
a frame comprising a plurality of members for support and each side of the frame is substantially open;
a vessel comprising a treatment chamber disposed in the frame for support thereof and the vessel comprises an opening on a top side configured to receive biomedical waste and a shredding system to treat the biomedical waste;
a circulation loop configured to provide water and ozone injection into the water into the treatment chamber and to receive water from the treatment chamber during a treatment cycle for the biomedical waste;
a cover rotatably connected to a member of the frame and configured to cover the opening in the vessel during the treatment cycle and non-use and to allow access to the opening during loading of the biomedical waste; and
a control system configured to monitor and control operation of the biomedical waste sterilization system, the control system comprising a verification/billing component, a shredder control component, and a User Interface.

20. The apparatus of claim 19, wherein the circulation loop comprises:
an ozone generator coupled to an ozone injector coupled to the treatment chamber;
a water pump coupled to the ozone injector;
a return pipe from the treatment chamber into the water pump, wherein the circulation loop runs from the treatment chamber to the water pump to the ozone injector and back to the treatment chamber.
